(19) United States
(12) Patent Application Publication
(10) Pub. No.: US 2025/0339311 A1
(43) Pub. Date: Nov. 6, 2025

(54) TEMPERATURE REGULATED FLUID THERAPY

(71) Applicant: Neurointact, Inc., Baltimore, MD (US)

(72) Inventors: Felino V. Cortez, JR., Bowie, MD (US); Bryan Nicholson, Annapolis, MD (US); Charly Busey, Easton, MD (US); Marc Mckenna, North Potomac, MD (US)

(21) Appl. No.: 18/652,653

(22) Filed: May 1, 2024

(51) Int. Cl. A61F 7/12; A61F 7/00

(52) U.S. Cl. CPC A61F 7/12; A61F 2007/001; A61F 2007/006; A61F 2007/0063; A61F 2007/0086; A61F 2007/0095

(57) ABSTRACT

A method for controlling tissue temperature involves providing a nasal cannula including a first elongated prong with an axial fluid channel and a downwardly-curved shape. The nasal cannula further includes a first aperture on a side wall of a distal portion of the first elongated prong. The first aperture provides access to the axial fluid channel. The method includes inserting the first elongated prong into a nasal cavity of a subject to face the first aperture at a pharyngeal wall of the subject and modifying a temperature of the pharyngeal wall by delivering a first fluid portion through the axial fluid channel and out of the first aperture to the pharyngeal wall.

### BACKGROUND

This disclosure relates to systems and methods for physiological temperature control. In some clinical settings, the management of the temperature of bodily organs and systems, such as the brain, can reduce the risk of injury, such as following certain types of trauma or medical events.

### SUMMARY

Described herein are systems and methods to change the temperature of a target area of a subject's body. A temperature control device may facilitate the cooling of the brain to mitigate brain damage due to injury or a medical procedure.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1A shows a nasal cannula assembly in accordance with one or more embodiments.

FIG. 1B shows an embodiment of the nasal cannula with two elongated prongs where at least one of the elongated prongs includes a first aperture on a side of the elongated prong and a second aperture on a distal end of the elongated prong.

FIG. 1C shows an embodiment of the nasal cannula from FIG. 1B with an elongated prong inserted into the nasal cavity of a subject.

FIG. 2 is a schematic diagram of a temperature control system in accordance with one or more embodiments.

FIG. 3A shows an example of the anatomy and internal systems of a subject.

FIG. 3B is a rear cross-sectional view of an example head of a subject.

FIG. 3C is an illustration of the circle of willis portion of a circulatory system and an illustration of a bottom view of a brain showing the circle of willis.

FIG. 4 shows a side cross-sectional view of a respiratory system of an example subject, as well as a perspective view of an example nasal cannula in accordance with one or more embodiments.

FIG. 5 is an illustration of an elongated prong of a cannula in accordance with one or more embodiments.

FIG. 6 shows a cannula inserted into a cavity of a subject in accordance with one or more embodiments.

FIG. 7 is a flow diagram of a process for modifying the temperature of one or more target areas of a subject in accordance with one or more embodiments.

FIG. 8 shows a cannula inserted into a cavity of a subject in accordance with one or more embodiments.

FIG. 9 is a flow diagram of a process for modifying the temperature of one or more target areas of a subject in accordance with one or more embodiments.

FIG. 10A shows a nasal cannula assembly in accordance with one or more embodiments.

FIG. 10B shows the nasal cannula assembly of FIG. 10A with tubing that can be connected using a tube connector in accordance with one or more embodiments.

FIG. 10C shows the nasal cannula assembly of FIGS. 10A and 10B with the fluid tubing connected in accordance with one or more embodiments.

FIG. 11A shows a nasal cannula including modular prongs in accordance with one or more embodiments.

FIGS. 11B, 11C, and 11D show respective modular prong structures in accordance with one or more embodiments.

FIG. 12A shows a nasal cannula including modular prongs in accordance with one or more embodiments.

FIGS. 12B, 12C, and 12D show respective modular prongs in accordance with one or more embodiments.

FIG. 13A shows an insulating sleeve that is configured to surround a fluid supply tube in accordance with one or more embodiments.

FIG. 13B shows a fluid supply tube partially covered by an insulating sleeve in accordance with one or more embodiments.

FIG. 13C shows a nasal cannula assembly, including a fluid supply tube covered by an insulating sleeve connected to a nasal cannula in accordance with one or more embodiments.

FIG. 14A shows a nasal cannula assembly, including a fastening device in accordance with one or more embodiments.

FIGS. 14B and 14C show respective examples of fastening devices in accordance with one or more embodiments.

FIG. 15 shows a single-prong nasal cannula in accordance with one or more embodiments.

FIG. 16A shows a cannula including a fluid-delivery prong and a temperature-measurement prong in accordance with one or more embodiments.

FIG. 16B is a close-up view of the temperature-measurement prong shown in FIG. 16A in accordance with one or more embodiments.

FIG. 17 is a flow diagram illustrating a process for implementing adaptive temperature management using a nasal cannula in accordance with one or more embodiments.

FIG. 18A shows a nasal cannula prong, including insertion depth markings in accordance with one or more embodiments.

FIG. 18B shows the nasal cannula prong of FIG. 18A inserted into a nasal passage of a subject in accordance with one or more embodiments.

FIGS. 19A and 19B show perspective and cross-sectional views, respectively, of a nasal cannula prong, including one or more rib features in accordance with one or more embodiments.

FIG. 20 shows an embodiment of the nasal cannula that includes a first elongated prong with an aperture on a distal end of the first elongated prong and a second elongated prong with an aperture on the side of the second elongated prong.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise therefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations, in turn, in a manner that may be helpful in understanding certain embodiments; however, the order of description should not be construed to imply that these operations are order-dependent. Additionally, the structures, systems, and/or devices described herein may be embodied as integrated components or as separate components. Certain aspects and advantages of these embodiments are described herein for the purposes of comparing various embodiments. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between the element(s)/structures(s), as illustrated in the drawings. It should be understood that spatially relative terms are intended to encompass different orientations of the element(s)/structures(s), in use or operation in addition to the orientations depicted in the drawings. For example, an element/structure described as “above” another element/structure may represent a position that is below or beside such other element/structure with respect to alternate orientations of the subject patient or element/structure, and vice-versa.

Certain reference numbers are re-used across different figures of the figure set of the present disclosure as a matter of convenience for devices, components, systems, features, and/or modules having features that are similar in one or more respects. However, with respect to any of the examples disclosed herein, the reuse of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. The use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Methods and structures disclosed herein for treating a patient also encompass analogous methods and structures performed on or placed on a simulated patient, which is useful, for example, for training, demonstration, procedure, device development, and/or the like. The simulated patient can be physical, virtual, or a combination of physical and virtual. A simulation can include a simulation of all or a portion of a patient, for example, an entire body, a portion of a body (e.g., nasal cavity), a system (e.g., respiration system), an organ (e.g., brain), or any combination thereof. Physical elements can be natural, including human or animal cadavers, or portions thereof, synthetic, or any combination of natural and synthetic. Virtual elements can be entirely in silico, or overlaid on one or more of the physical components. Virtual elements can be presented on any combination of screens, headsets, holographically, projected, loudspeakers, headphones, pressure transducers, temperature transducers, or using any combination of suitable technologies.

The present disclosure relates to systems, devices, and methods for targeted temperature control of one or more body parts. Although certain aspects of the present disclosure are described in detail herein in the context of nasal, cerebral, and/or lung anatomy, devices, and procedures, it should be understood that such context is provided for convenience and clarity, and the concepts disclosed herein are applicable to any suitable anatomy, devices, and medical procedures.

In some aspects, the present disclosure relates to medical devices for changing/modifying the temperature of one or more anatomical areas, tissues, and/or organs of a human or animal subject. In some applications, devices disclosed herein can be used to reduce the temperature of a subject's brain, which can be desirable to slow down certain metabolic processes following certain types of trauma or medical events. Temperature regulation as disclosed herein, can advantageously reduce inflammation that can cause further damage to brain tissue, decrease oxygen demand by slowing the metabolic rate, limit ischemic injury, protect against reperfusion injury, prevent brain swelling and increased intracranial pressure, and/or otherwise improve a subjects physiological condition in some situations. Implementation and utilization of devices and processes disclosed herein can be useful as a treatment for cardiac arrest, neonatal hypoxic-ischemic encephalopathy (HIE), stroke, traumatic brain injury, and/or other conditions.

Aspects of the present disclosure may advantageously be implemented in connection with therapeutic hypothermia to achieve and/or maintain body and/or brain temperatures between about 32-36° C. (89.6-96.8° F.). In some cases, it may be desirable to implement therapeutic hypothermia in accordance with the present disclosure relatively early in the therapeutic window following a traumatic injury/event, such as less than about 30 minutes to about 6 hours after injury, as a means to delay necrotic cell death and apoptotic cell death. This may lead to positive effects including, among other things, a lower cerebral metabolism, which reduces harmful metabolic byproduct build-up resulting from inadequate blood flow, reduced cerebral oxygen requirements, prevention of neurogenic fever, reduced intracranial pressure (ICP) encephalitis, and the like.

The present disclosure relates to inventive nasal cannula designs that advantageously improve patient comfort, safety, ease of use, efficiency, cost reduction and provide benefits relative to other solutions. Certain aspects of the present disclosure are presented herein in the context of a patient being treated in a medical facility, though it should be understood that embodiments of the present disclosure can be implemented in a variety of healthcare settings. Cannula systems, assemblies, and devices disclosed herein can include and/or relate to a cannula configured to be inserted into a cavity of a human or animal subject. In some embodiments, the cannula includes at least one elongated prong that is shaped to be inserted into one or both of the nostrils/nares of a patient and advanced into the nasal cavity. A side wall of the cannula comprises and/or has formed therein one or more apertures through which fluid may be directed to a target area within the target cavity. Accordingly, the fluid may be directed to a specific area for a targeted temperature treatment. In some embodiments, a cannula is configured to deliver a temperature-controlled fluid or fluid at a flow rate to effectuate a heat exchange with the target area. The term “aperture” is used herein according to it's broad and ordinary meaning, and may refer to any opening, hole, gap, orifice, pore, vent, slot, window, port, or fenestration through which fluid can pass to exit from within a lumen of a structure (e.g., tubular cannula prong) to an area external to the structure.

In some implementations, a nasal cannula of the present disclosure includes an elongated prong through which fluid (e.g., air, oxygen or mixture of breathing gases) can flow from a proximal portion to a distal portion (e.g., end, tip) of the prong. The term “prong” is used herein according to its broad and ordinary meaning, and may refer to any shape, length, or design of a tube, shaft, nozzle, probe, or other type of projection or elongate member configured to be inserted into a target space or cavity. Nasal cannula prongs disclosed herein can have a proximal end portion that is fluidly coupled to a core or base, allowing air or other fluid to pass from the base/core into the body of the prong, such as into a fluid channel/lumen of the prong that spans between a proximal fluid inlet and a distal aperture. The distal end of nasal cannula prongs disclosed herein, in some embodiments, the axial distal end of the prong is closed-off/sealed, whereas a dorsal side opening/aperture near the distal end/tip may be provided to serve as a port for fluid egress. Fluid flowing through the prong may escape the dorsal side aperture. Fluid may flow into the flow channel/lumen of the prong from the proximal base/core associated therewith toward the distal end of the prong. As the fluid exits the prong, the fluid may be directed from the prong in a direction approximating the average surface normal over an area of the opening.

Advantages over the technical limitations and drawbacks of contemporary technology include that the disclosed subject matter is relatively easy and quick to implement. In accordance with some implementations disclosed herein, a medical various preferences may be set for a temperature-controlled fluid directed through one or more prongs of a nasal cannula to cool the subject. This has advantages over solutions that require that a subject be intubated due to the discomfort of having cold air blown into their nasal cavity. Instead, the disclosed subject matter provides a safe, practical, and effective method for delivering cool fluid to the nasal cavity. Accordingly, the disclosed subject matter provides solutions that are relatively comfortable and tolerable for awake subjects. Further, the disclosed subject matter provides an efficient approach to delivering cold air to promote selective brain cooling and induce rapid initiation of cerebral cooling.

Nasal cannula prong design, in accordance with aspects of the present disclosure, can advantageously allow for local application of cold fluid directly to a target area within the target cavity (e.g., nasal cavity). In embodiments in which the cannula comprises nasal prongs configured to cool a targeted area in a nasal cavity, blowing cool air/fluid through the aperture in the nasal prongs may induce rapid initiation of cerebral cooling in the human or animal subject.

In some embodiments, the target area in the nasal cavity is in close proximity or adjacent anatomically to the cavernous sinus and internal carotid artery, cavernous sinus, the circle of willis, and cerebrospinal fluid in the basal cistern, which circulates in the brain. In some embodiments, cold fluid flow through the prong can effectively maintain a constant target temperature in the human or animal subject in order to produce a clinically desired significant brain temperature reduction. This may be used in cases where brain swelling is a significant issue.

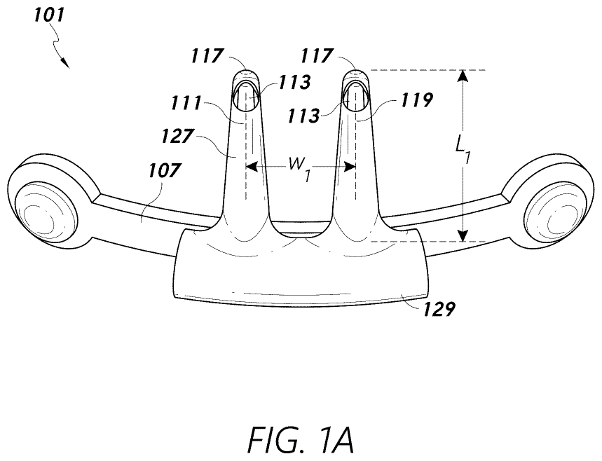

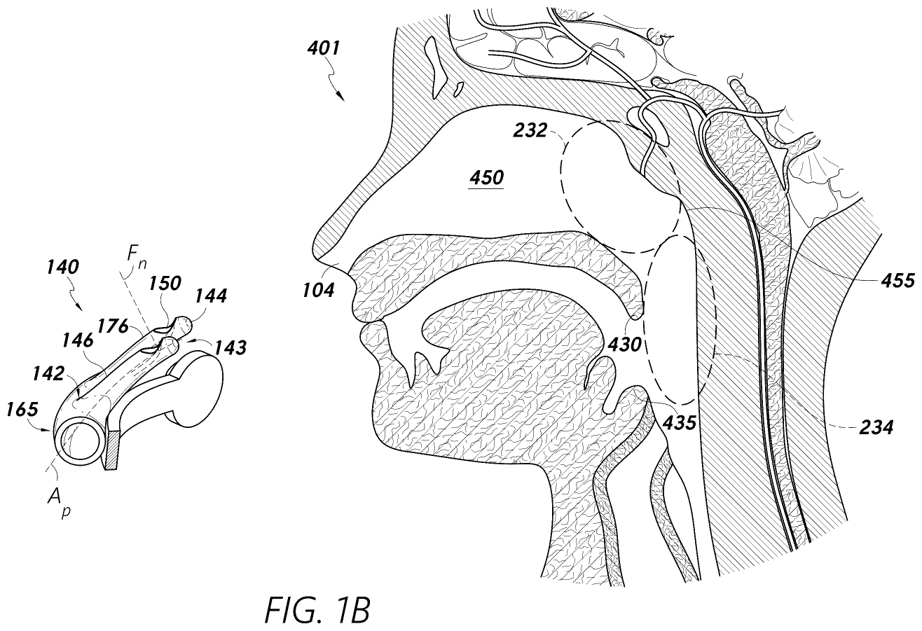

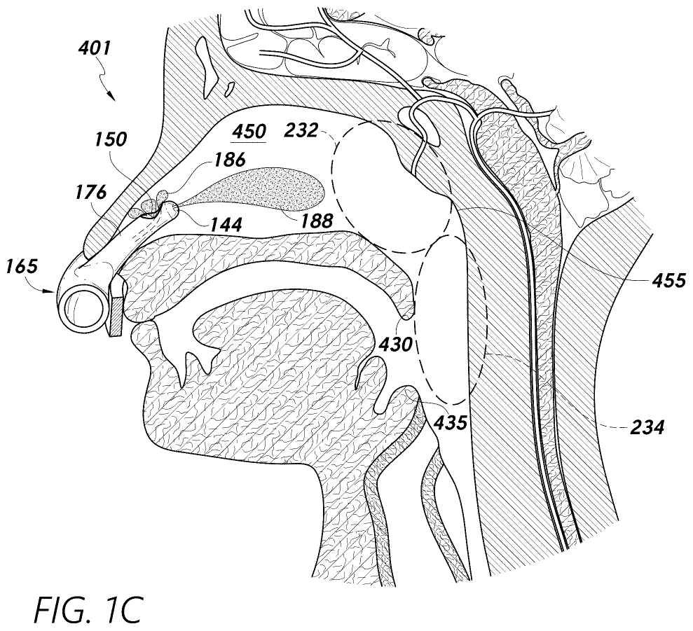

FIG. 1A shows a nasal cannula assembly 101 in accordance with one or more embodiments. The nasal cannula assembly may be affixed to a cavity of a subject to direct a fluid into the cavity. Fluid directed through the nasal cannula assembly 101 may be directed to a precise location inside a cavity of the subject to effect a change of the location to treat or otherwise benefit the subject. In various embodiments, the nasal cannula assembly 101 is configured to modify temperature of a location inside a cavity of the subject.

The nasal cannula assembly 101 may include one or more elongated prongs 127 that are configured to direct a fluid through a cavity of a subject. The nasal cannula assembly 101 may include a support structure 107 configured to attach the nasal cannula assembly 101 to a subject. Attached to the support structure 107 is a fluid supply connector hub 129. Fluid may be directed through the fluid supply connector hub 129 and into the one or more elongated prongs 127. In an example embodiment, the nasal cannula assembly 101 includes a first elongated prong 127. In another example embodiment, which is shown in FIG. 1A, the nasal cannula assembly 101 also includes a second elongated prong 119.

At least one of the one or more elongated prongs may include an aperture to which fluid is directed from the nasal cannula assembly 101 to the subject. In an example embodiment, at least one of the elongated prongs includes a side aperture 113 on a side of the elongated prong. The aperture may direct fluid in a direction that is approximately perpendicular to a longitudinal axis 111 of the elongated prong. Accordingly, fluid that exits the side aperture 113 may be directed to a side or wall of a cavity instead of being directed deeper into a cavity.

In an example embodiment, at least one of the elongated prongs may include a distal aperture 117 that is configured to direct fluid that flows through the nasal cannula assembly 101 directly into a cavity or other opening of a subject rather than directing the fluid at an angle such as when the fluid is directed through the side aperture 113. In an example embodiment, a distal end of at least one of the elongated prongs is sealed and all fluid is directed through the side aperture 113. Alternatively, at least one of the elongated prongs may include a distal aperture 117 without other apertures for fluid to exit the nasal cannula assembly 101.

In the embodiment of the nasal cannula assembly 101 shown in FIG. 1A, the nasal cannula assembly 101 may be configured to be affixed to a head of the subject with the two elongated prongs inserted into the nasal cavity through the two nares of the subject. The nasal cannula assembly 101 may be shaped with an inter-prong distance w₁ between the elongated prongs that corresponds to a distance between the nares of the subject. The prong length L₁ of the elongated prongs may be configured to extend into the nasal cavity to a target area such as a wall of a oropharynx or nasopharynx of the subject. The inter-prong distance w₁ and prong length L₁ may be modified based on the needs of the subject.

FIG. 1B shows an embodiment of the nasal cannula 140 with two elongated prongs 146 where at least one of the elongated prongs 146 includes a first aperture 150 on a side of the elongated prong and a second aperture 144 on a distal end 143 of the elongaged prong 146. The nasal canula 140 may be configured to be affixed to the head of a subject 401. The nasal cannula 140 may include a connector hub 165 to which one or more elongated prongs are connected to the connector hub 165 at a base 142 of the one or more elongated prongs 146.

Each of the one or more elongated prongs 146 may be configured to be inserted through the nares 104 of the subject 401 to direct a fluid into the nasal cavity 450. In various embodiments, the nasal cannula 140 includes an elongated prong 176 that is configured to be inserted into a second nare of the subject 401 such that both elongated prongs are inserted concurrently into the subject 401. In various environments, one of the elongated prongs 146 may include a first aperture that is configured to direct fluid in a fluid egress direction Fₙ perpendicular to a longitudinal axis Aₚ of the elongated prong. In some embodiments, at least one of the elongated prongs 146 may include a second aperture 144 at a distal end 143 of the elongated prong that is configured to direct fluid in a direction parallel to the longitudinal axis Aₚ of the elongated prong.

FIG. 1C shows an embodiment of the nasal cannula 140 from FIG. 1B with elongated prongs inserted into the nasal cavity 450 of a subject 401. The nasal cannula 140 is affixed to the head of the subject 401 such that the elongated prong 176 penetrates the nare 104 into the nasal cavity 450. Fluid that is directed through the elongated prong 176 will not interact with any portion of the subject until the fluid exits at least one of the apertures. As shown in the embodiment, the elongated prong 176 includes a first aperture 150 that directs fluid 186 into a wall of a nasal cavity 450. The prong 176 also includes a second aperture 144 that directs a fluid 188 deeper into the nasal cavity 450.

Fluid that is directed through the first aperture 150 may interact and modify one or more physical characteristics of the subject at the location that the fluid exits the nasal cannula 140. One more physical characteristics may include a temperature, humidity, and pressure. Fluid that is directed through the second aperture 144 may interact and modify one or more physical characteristics at a separate location from the fluid that is directed through the first aperture 150. The length of the elongated prong 176 may be adjusted based on the needs of the subject by swapping elongated prongs from the nasal cannula assembly 101. In another example, a size and location of the aperture to direct fluid at a target location of interest may be adjusted by swapping elongated prongs with the desired aperture.

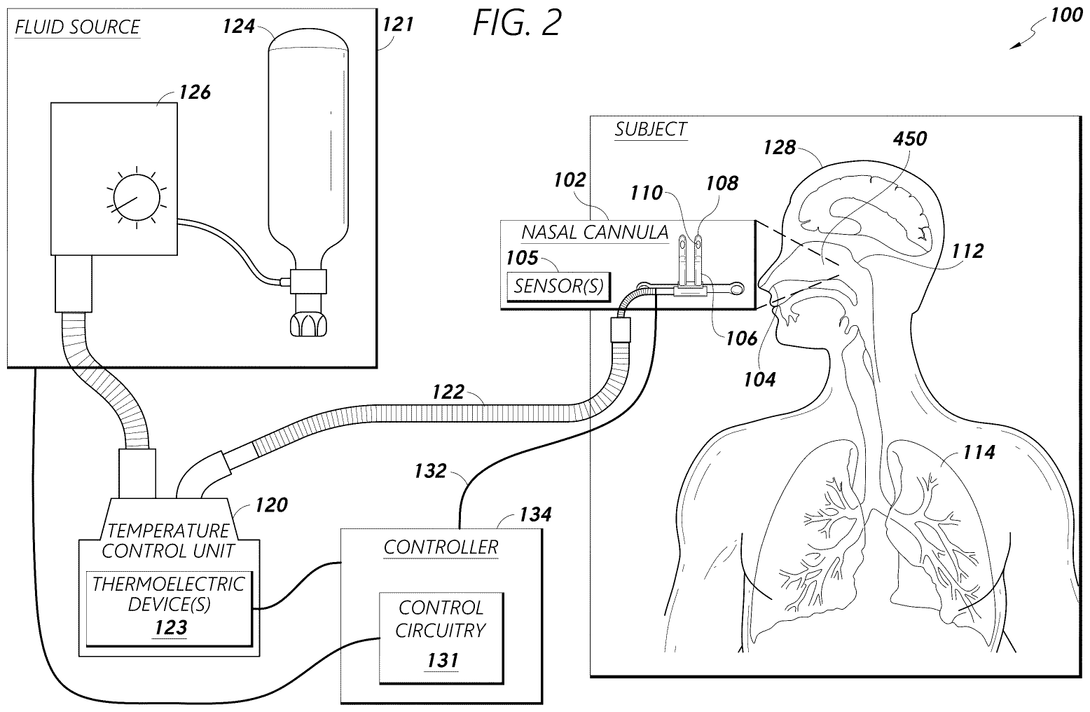

FIG. 2 is a schematic diagram of an embodiment of a temperature management system 100 for controlling the temperature of a subject. The temperature management system 100 is configured to deliver a temperature-controlled fluid to a target area 112 within a cavity of a subject. Although some illustrations and diagrams in this disclosure depict the device used in a nasal cavity 450 of a subject 128, the disclosed subject matter is not limited to use on human beings, and the disclosed cannula is not limited to use in a nasal cavity. As used herein, the term “fluid” may refer to a gas, a liquid, or a combination thereof. That is, the term “fluid” is used herein according to its broad and ordinary meaning and may refer to any substance or mixture of substances that exhibits the ability to flow and conform to the shape of its container under normal conditions of use. This includes, but is not limited to, liquids, gases, plasmas, colloids or mixtures of gases and liquids. The term encompasses substances that can change their state under varying temperatures, pressures, or compositions encountered during the use of the claimed invention. It further includes both Newtonian and non-Newtonian fluids, where Newtonian fluids maintain a constant viscosity under varying shear rates, and non-Newtonian fluids exhibit a change in viscosity with changes in shear rate. This definition is intended to cover all phases and states of matter that possess the characteristic fluidity necessary for the operation or application of the described invention, including but not limited to applications involving fluid dynamics, fluid transport, fluid storage, or fluid manipulation technologies.

In the example embodiment shown in FIG. 2, the temperature management system 100 comprises a temperature control unit 120 that is configured to direct fluid at a set temperature, flow rate, and/or mixture of substances through a tube 122 to a cannula 102. The cannula 102 is shaped to be inserted into the nasal cavity 450 of the subject 128 through the nares 104. The temperature control unit 120 can advantageously regulate the temperature of the fluid prior to the fluid being directed through an axial fluid channel of one or more prongs of the cannula 102.

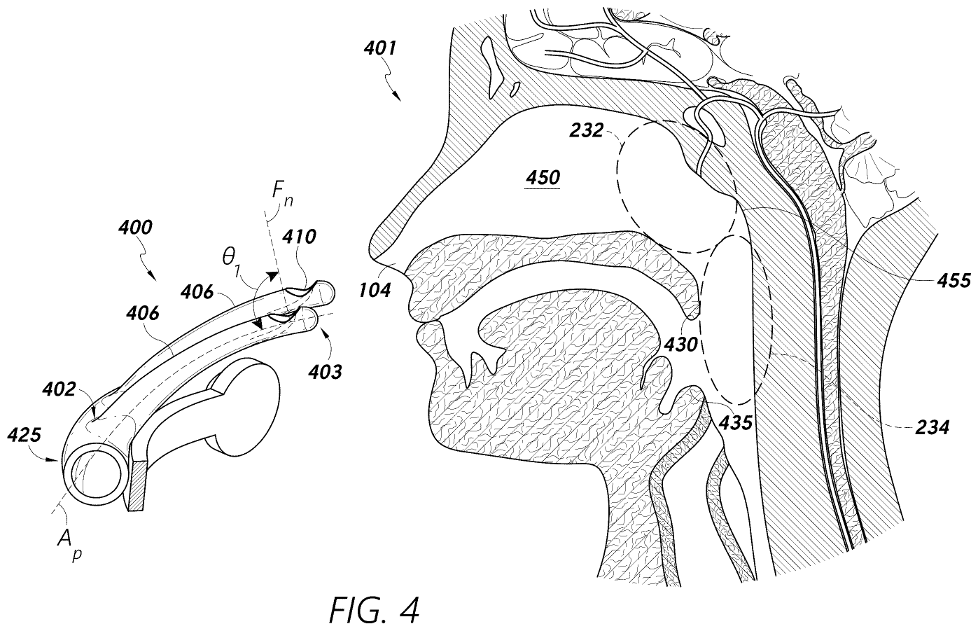

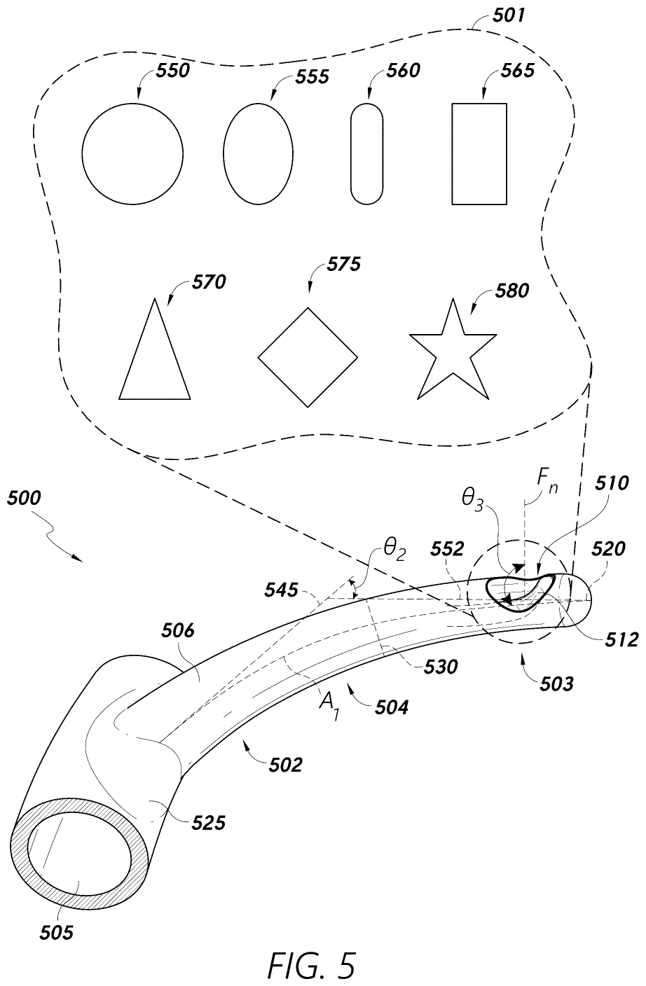

Once inserted, the cannula 102 may deliver the temperature-controlled fluid by bypassing a significant portion of the nasal cavity 450 to a specific target area 112 to control/modify the temperature (heat, cool, or maintain) of the target area 112 with minimal temperature variation to the other surrounding tissue within the nasal cavity. Accordingly, secondary cooling effects may be mitigated in view of design characteristics of the cannula 102 and the temperature management system 100. In some embodiments, the cannula 102 includes one or more prongs 106, which have an aperture 110 on the back or dorsal side thereof. When the cannula 102 is inserted into the nasal cavity 450, the aperture 110 can be positioned and configured to direct the temperature-controlled fluid to the target area 112 on a wall (e.g., roof, back) of the nasal cavity 450. In the example embodiment of the cannula 102 shown in FIG. 2, the distal end 108 of the prong(s) 106 is sealed or closed to direct the flow of the temperature-controlled fluid through the aperture 110. In some embodiments of the cannula, the distal terminal end may not be closed off (as shown in FIGS. 4 and 5).

In some implementations, the cannula prong(s) 106 is/are configured as extended prongs configured to penetrate relatively deep into the nasal cavity. Such extension depth can prevent, or reduce the risk of, tissues of the nasal cavity exchanging heat and increasing the fluid temperature to an undesirable degree before the fluid exits the prong(s) 106. The extended prong also blocks the delivery of cold fluid to the interior surface of the ala nasi and dorsum nasi, which comprise an outer structure of the nose. Blowing temperature-controlled air through the extended prong 106 also prevents temperature-controlled fluid from entering the nasolacrimal duct and the eyes.

In some embodiments, the present disclosure relates to a nasal cannula 102 with a connector hub, or base, with first and second elongated nasal prongs extending therefrom. The prong(s) may be designed to extend through the nares of the human or animal subject, terminating in the area of the oropharynx or nasopharynx when it is completely inserted into a nasal passage. The distal ends 108 of the prongs 106 can be rounded and sealed. Air may be inserted through a dorsal aperture near a distal end 108 of the prong(s) 106. The prongs 106 can advantageously be contoured to adapt to a nasal cavity and accommodate a curved path from the nares to the oropharynx. Although two prongs 106 are shown and described in some contexts, it should be appreciated that in some implementations, nasal cannulas of the present disclosure consist of only a single prong.

The prongs 106 can have a smooth surface with few or no sharp edges. The prongs 106 can be soft, or semirigid or rigid to maintain a specific tube stiffness and wall integrity during insertion into the nasal cavity. The aperture or aperture can be round, oval, elliptical, rectangular, triangular, square, diamond shape, or other geometrical shapes. The inner lumen of the nasal prongs provides a clear and free passage of the fluid from the nasal cannula body to the exit port or aperture of the nasal prongs. In some embodiments, a flexible corrugated supply tubing is coupled to the nasal cannula to receive fluid flow. In some embodiments, the cannula has extensive wing features on the sides with terminal ends designed for detachable head straps. The nasal cannula 102 may comprise any suitable or desirable materials, such as any synthetic, semi-synthetic, and/or organic compounds, including, e.g., polyvinyl chloride (PVC), polyurethane, polyethylene, polycarbonate, polyethylene terephthalate, high-density polyethylene (HDPE), polystyrene, polymethyl methacrylate or other medically approved materials.

Temperature-controlled fluid may exchange heat with the target area 112, which can heat or cool the fluid to approach the body temperature of the human or animal subject as the fluid enters the lungs 114. In some embodiments, one or more sensors 105 may be integrated or otherwise associated with the cannula 102. For example, the sensor(s) 105 may be disposed on and/or in one or more of the prongs 106 as to be insertable into the cavity or other portion of the subject 128 with the prong(s) 106 to perform a measurement. The temperature, flow rate, mixture, or other properties of the fluid may be adjusted based on or in response to the measurements from the sensor(s) 105. For example, the sensor(s) 105 may be temperature sensor(s) configured to measure the temperature inside the nasal cavity 450. The temperature of the fluid, as controlled by the temperature control unit 120, may be adjusted based on the measurement communicated to the temperature control unit 120 by a circuit 132.

In some embodiments, a fluid source system 121 may supply a mixture of gas(es) and/or liquid(s) to a fluid blender 126 (e.g., air oxygen blender and the like) that is configured to provide a set mixture of gases or a mixture of fluid and gas. The mixture or composition of certain breathing gas(es) (e.g., air and oxygen) from the fluid blender 126 may be delivered to a heating or cooling device, such as the temperature control unit 120, which can be configured to heat/cool and/or humidify the air mixture according to a set point. The term “mixture” is used herein according to its broad and ordinary meaning and may refer to a percent composition of gases in the air or other fluid. An example of a gas mixture is Heliox (a mixture of Helium and Oxygen, or Nitric oxide (a mixture of Oxygen and nitrogen.

In an example embodiment, the fluid may comprise a saline solution. A saline solution may be a composition of sodium chloride and water. In various embodiments, the saline solution may comprise a mixture of about 0.9% sodium chloride in water by mass. In an example embodiment, the fluid may comprise perfluorocarbons, which are chlorinated hydrocarbons. An example of a perfluorocarbon is perfluorooctane, which has the chemical formula C₈F₁₈. However, perfluorocarbon may not be preferable due to environmental concerns, and/or due to various other adverse effect on organic tissue that can result from use of perfluorocarbons.

In some embodiments, the fluid may comprise xenon gas. In some embodiments, the fluid may comprise atmospheric air, which is a mixture of gases comprising approximately 78% nitrogen, 21% oxygen, and small amounts of other gases such as carbon dioxide. In some embodiments, the fluid may comprise a phase change material configured to change phases between a solid, liquid, or gas at one or more points during the heat exchange process. For example, the fluid may comprise perfluorodecalin, with a chemical structure C₁₀F₁₈, which may change phase during the process of controlling a physiological temperature.

In some embodiments, one or more mechanisms that provide fluid to the cannula 102 include a controller 134 that automatically adjusts one or more properties of the fluid using certain control circuitry 131. In some embodiments, the controller 134 may adjust one or more properties of the fluid based on one or more measurements received from the subject 128. Any of the processes and/or functionality described herein may be performed at least in part by the control circuitry 131, which may be embodied in a single device, or across multiple devices, which may be communicatively coupled over wired and/or wireless connection(s). For example, the control circuitry 131 may be embodied in whole or in part in any of the components of the temperature management system 100, including the fluid source system 121, the temperature control unit 120, the controller 134, the nasal cannula 102, or any other device or system. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry 131 comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The controller 134 may be configured to receive instructions to adjust one or more properties of a fluid to be delivered to the subject 128. The controller may execute instructions such as an electronic signal causing one or more devices to adjust the property of the fluid being delivered. For example, controller 134 may be configured to transmit signals to the temperature control unit to adjust a set point temperature or the flow rate of the fluid being delivered to the subject 128.

Temperature control devices and systems disclosed herein can be configured to deliver fluid at a wide range of temperature and flow rates. For example, a fluid source system 121, which may also be considered to include the temperature control unit 120 and/or controller 134, may set a fluid temperature, fluid flow rate, fluid mixture, and similar fluid properties to be delivered to the subject. For example, fluid source systems that may be implemented in accordance with various embodiments may include one or more fluid tanks 124 (e.g., compressed fluid tanks) connected to regulator(s) to control the pressure of the fluid leaving the fluid tank(s) 124. The fluid source system 121 may further include an oxygen mixer and a humidifier. The fluid source system 121 may comprise a heater/cooler, such as a thermoelectric element. The fluid source system 121 may be electrically controlled and connected to a controller 134 that processes instructions to adjust the fluid flow rate, temperature, fluid blender 126, or other properties. Controller 134 may receive one or more measurements, such as a temperature measurement, and automatically adjust one or more fluid flow properties based on the measurement.

In some embodiments, the controller 134 may receive one or more measurements from the cannula 102 and/or sensor(s) 105 associated therewith. The controller 134 may adjust one or more properties of the fluid based on measurements received over connection/circuit 132. In some embodiments, the circuit 132 may transmit temperature measurements of the subject 128 to the controller 134. In response, the controller 134 may execute an instruction to adjust a flow rate, fluid temperature, fluid mixture, or the like of the fluid flowing to the subject 128. In some embodiments, the flow rate of the fluid may be adjusted via an air regulator that is connected to a fluid source such as a fluid tank.

In some embodiments, the temperature control unit 120 may heat or cool the fluid using one or more thermoelectric devices 123. The thermoelectric device(s) 123 can comprise solid-state device(s) configured to convert electrical energy directly into thermal energy, such as through the Peltier effect. Use of the thermoelectric device(s) 123 to cool fluid for cerebral cooling in accordance with embodiments of the present disclosure can involve applying a direct current to a thermoelectric module, thereby causing heat to be absorbed from one side of the module and released on the other side. This process can create a temperature differential, with one side becoming cooler than the ambient temperature and the other side becoming warmer. The cool side can be used to lower the temperature of the air or fluid passed over the cool side of the device, such as by a fan or some other means, thereby cooling the fluid.

In some embodiments, a mixture of the fluid may be controlled by a fluid blender 126 that controls the oxygen content of the fluid. In some embodiments, the fluid mixtures may include other gases, such as nitrogen or helium. In some embodiments, the temperature control unit 120 may include a fluid tank 124 of sterile water.

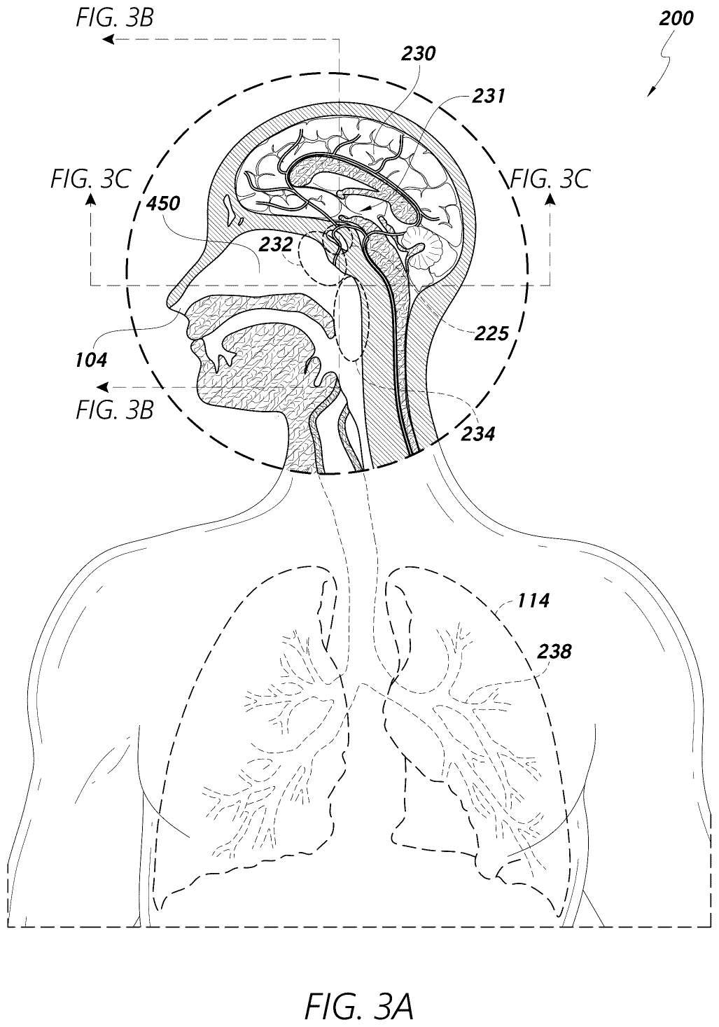

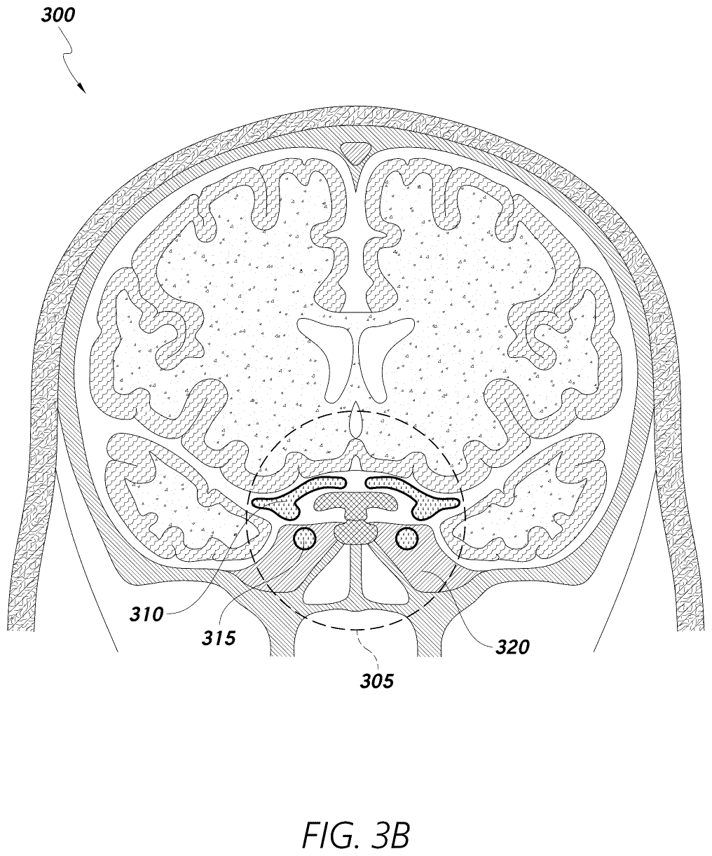

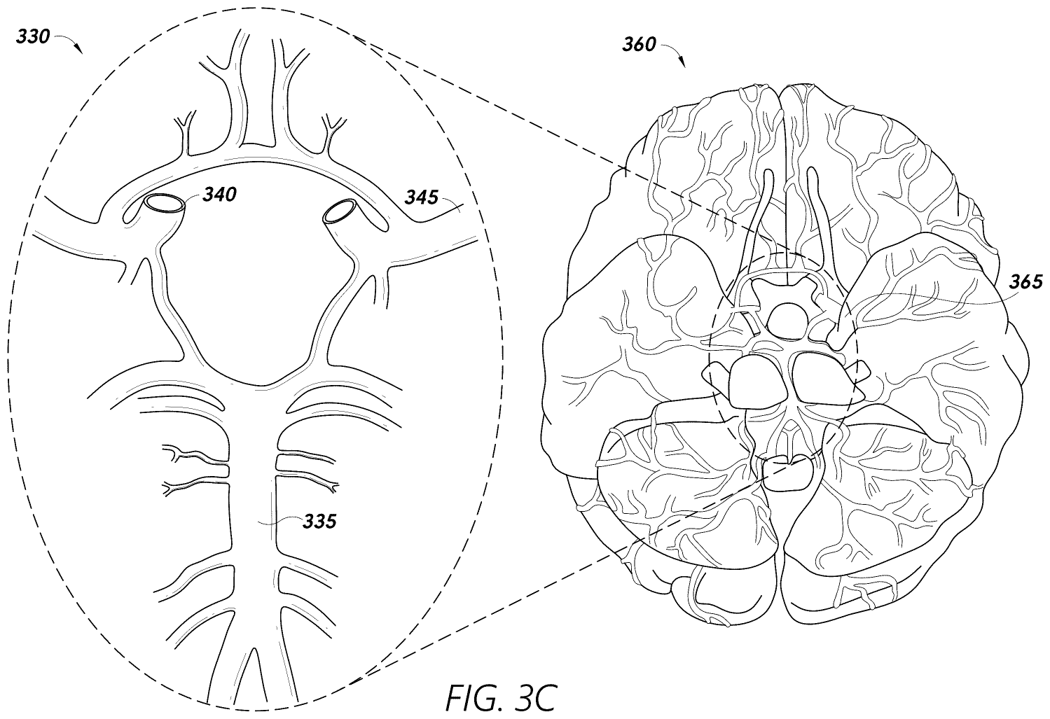

FIG. 3A is an illustration 200 of internal systems, including the nervous system, respiratory system, and circulatory system and an upper portion of a subject. In some embodiments, the disclosed subject matter may be configured to deliver temperature-controlled fluid through the respiratory system to change the temperature of a specific organ.

For example, the specific organ may be the brain. Accordingly, one way to bring about a temperature change in the brain may be to direct temperature-controlled fluid to a target area 225 in the nasal cavity 450, which will efficiently distribute the temperature change to the rest of the brain. In some embodiments, the target area 225 can be in the nasopharynx 232 or oropharynx 234 of the nasal cavity 450 in close proximity or adjacent to the cavernous sinus and internal carotid artery, the circle of willis, and cerebrospinal fluid in the basal cistern, which circulates in the brain. The hypothalamus 231 may also receive the cooling effect from the temperature-controlled fluid. After providing sufficient cooling effect, the hypothalamus 231 may be reset. Here, when the temperature of the target area 225 is reduced by the temperature-controlled fluid at varying flow rates, the arterial and venous blood's temperature may be changed, and the circulating blood could, in turn, change the temperature of the cerebrum. Lung 114 and trachea, bronchi, and bronchi tree 238 are shown in FIG. 3A. For the purposes of this disclosure, “close proximity” refers to a spatial relationship between two components or elements of the disclosure wherein the distance separating said components or elements is between about 1 millimeter (mm) to about 10 millimeters (mm), measured in a straight line from the nearest point of one component to the nearest point of the other component (or part of a subject or organ), irrespective of their orientation. This definition is intended to specify the precise range of distances within which certain interactions, effects, or functionalities of the disclosure are achieved, operational, or optimized, as detailed in the claims and descriptions herein.

The cannula 102 is inserted through the nares 104 to transport the temperature-controlled fluid through the nasal cavity without interacting with any tissues until it reaches the target area 225. The target area may be selected based on its ability to distribute the temperature change (heating or cooling) through one or more organs in the body. The target area 225, shown in FIG. 3A, comprises various anatomical features such as the internal carotid artery, which will efficiently distribute heat or cold exchanged from the temperature-controlled fluid to the rest of the brain 230. In some embodiments, the target area may be another part of the human or the animal subject, such as but not limited to, lungs, liver, intestines, kidneys or the like.

FIG. 3B is an illustration 300 of a cross-section of a brain. In some embodiments of the disclosed subject matter, a temperature-controlled fluid may be delivered to a target area that is near or adjacent to major blood vessels that distribute blood to the brain. Accordingly, heat exchange at the target area may be used to regulate the temperature of the brain effectively.

In an example embodiment, heat exchange between the temperature-controlled fluid and a wall of a cavity in the subject through conduction as heat flows from a warmer object to another object via direct contact. For example, heat energy may flow from a wall of the nasal cavity to a cold fluid that comes into contact with the wall. The flow of heat will cause the tissues surrounding the wall to gain or lose heat energy as heat energy flows to or from the surrounding tissues to the wall. Heat exchange will also occur in blood within blood vessels or other bodily fluids, such as cerebrospinal fluid, that are in the surrounding tissues.

These aforementioned bodily fluids may propagate the heat transfer to other parts of the body. Bodily fluids that have exchanged heat with the temperature-controlled fluid may be distributed throughout the rest of a subject where the bodily fluids will exchange heat with tissues and the other parts of the subject. For example, cold blood and a blood vessel adjacent to a wall of the nasal cavity that is subjected to cooled fluid may be pumped through natural bodily processes throughout the brain to cool the brain. For instance, the brain of a subject may be cooled by inserting that the disclosed cannula into a nasal cavity of the subject and causing the cannula to blow cold fluid onto a target area that is adjacent to bodily fluids that are distributed to the brain. This may be useful for medical practitioners who intend to cool the brain to reduce brain swelling or other damage that may be mitigated by cooling the brain.

The target area may be adjacent to or near the major blood vessels of the brain, indicated, for example, by the blood vessels within the area 305. Temperature-controlled fluid that is directed to the target area may exchange heat or cool the tissue and blood within the middle cerebral artery 310, the posterior cerebral artery 315, or another artery within the area 305. In some embodiments, the target area may include cerebrospinal fluid in the basal cistern 320, which also circulates throughout the brain.

In some embodiments, one or more sensors adjacent or attached to the cannula may collect measurements from the target area. One or more properties of the temperature-controlled fluid may be adjusted based on the collected measurements. The adjustable properties of the temperature-controlled fluid may include, flow rate, temperature, the type of fluids used, the mixture of gas with liquid, etc. For example, the temperature of the brain around area 305 or another area of the brain may be measured and transmitted to the controller 134 to adjust one or more properties of the temperature-controlled fluid that is directed to the target area.

In an example of use, a property of the temperature-controlled fluid may be adjusted to increase a cooling effect in response to a high temperature in the brain. For instance, the temperature of the temperature-controlled fluid may be lowered in response to a high-temperature measurement or increased pressure in the cranial cavity or the brain. In another instance, the flow rate of the temperature-controlled fluid may be increased in response to a high-temperature measurement or increased pressure in the cranial cavity or the brain to increase a cooling effect.

FIG. 3C is an illustration of the circle of willis 330 portion of a circulatory system and an illustration of a bottom view 360 of a brain showing the circle of willis. The circle of willis 330 is a group of blood vessels near the obituary gland and the optic chiasm that supply a significant portion of blood to the brain. Accordingly, the circle of willis 330 is an ideal target to effectuate temperature control in the brain or the cranial cavity. The area of the nasal cavity nearest to the circle of willis 330 may be targeted to direct temperature-controlled fluid in order to effect temperature control on the rest of the brain. In order to reach the exterior of the cranial cavity, the cannula 102 is designed to have an infection of angle and length to reach the nasopharynx and oropharynx portion that is in close proximity or adjacent to the circle of willis. Moreover, the cannula has an aperture on the dorsal portion of the oropharynx so that the temperature-controlled fluid is directed to the oropharynx.

As shown in FIG. 3C, the circle of willis 365 comprises a group of blood vessels that include the internal carotid artery 340, the basilar artery 335, and the middle cerebral artery 345. The various arteries of the circle of willis 330 supply blood to the brain and form a continuous loop. As shown in FIG. 3C, the circle of willis 330 is prominently displayed from a bottom view of the brain and is adjacent to an area of the oropharynx in the nasal cavity. Fluid that is directed to a target area within the oropharynx may effectively exchange heat or cooling with blood flowing through the circle of willis 330, which will distribute the heat or cooling exchanged blood with the rest of the brain or cranial tissue.

FIG. 4 shows a side cross-sectional view of a respiratory system of an example subject 401, as well as a perspective view of an example nasal cannula 400. The nasal cannula 400 includes a plurality of prongs 406, each configured to be inserted in the nasal cavity 450 of the subject 401. The description below of a singular prong 406 may be understood to refer to either or both of the prongs 406 shown. The prong 406 may have an elongated, shaft-type form, and may comprise one or more apertures 410 on the side of the prong 406. The prong 406 of the nasal cannula 400 may each comprise a tube with a proximal end and a distal end. The aperture 410 of the prong 406, to which temperature-controlled fluid is directed through a lumen of the prong 406, may be positioned on the side of the tube.

The portion of the prong 406 between the proximal inlet end 402 and distal end 403 may be considered the side of the prong 406. A line Aₚ drawn between the proximal end and distal end of the tube is a longitudinal axis of the prong 406. In embodiments where the prong 406 is curved, the longitudinal axis Aₚ may follow the curve of the prong tube such that the longitudinal axis is substantially in the middle of the tube at all points of the tube. Accordingly, fluid directed down the tube/channel of the prong 406 from the proximal inlet end 402 to a distal end 403 can travel along the longitudinal axis Aₚ in a direction from the proximal inlet end 402 to the distal end 403. The aperture 410 can be positioned on a side of the respective elongated prong 406 that is opposite a curve of the axis Aₚ of the prong 406. Each prong 406 may be about 6-10 cm in order to reach the proximity of a target area. In some embodiments, the cooling effect from the prong 406 may enable the fluid or gas to cool the hypothalamus 231 in order to reset the hypothalamus 231. Resetting the hypothalamus refers to attempts or methods aimed at modifying or regulating its functions to improve health outcomes or address certain medical conditions. The hypothalamus is a small but crucial part of the brain that plays a key role in regulating many bodily functions, including temperature control, thirst, hunger, sleep cycles, emotional responses, and the endocrine system through its connection with the pituitary gland. By cooling the hypothalamus using the specially designed cannula, it might be possible to influence or “reset” some of the hypothalamus functions.

When the nasal cannula 400 is inserted in the nasal cavity 450 of the subject 401, the aperture 410 on the side wall of the prong 406 may direct fluid more efficiently into a wall or other tissue in the cavity instead of directing the fluid further down into the cavity of the lower respiratory tract. Accordingly, the fluid is directed in a substantially perpendicular direction to the longitudinal axis Aₚ. In embodiments where the tube of the prong 406 is tapered such that the proximal inlet end 402 and the distal end 403 have different diameters, the substantially perpendicular direction may be less than about 90° relative to the tangent direction of fluid traveling down the longitudinal axis Aₚ at a midpoint along the length of the prong 406. In some implementations, fluid may be directed from the tube at an angle between about 85° and about 95° relative to the longitudinal axis Aₚ at the lengthwise position of the aperture 410. In some embodiments, fluid is directed from the tube/prong 406 at an angle between about 80° and about 95° relative to the longitudinal axis Aₚ at the lengthwise position of the aperture 410.

In some embodiments, the aperture 410 may be considered to direct the fluid in a direction corresponding to a line Fₙ that is normal with respect to the opening of the aperture 410 and the axis Aₚ at the lengthwise position of the aperture 410. The fluid egress direction Fₙ may correlate to an average surface normal of the aperture 410. The surface normal Fₙ may be determined based on a direction pointing away from the prong 406, at a perpendicular angle θ₁ to a plane of the aperture and/or at a perpendicular angle θ₁ to the axis Aₚ at the lengthwise position of the aperture 410. In some embodiments where the aperture is in a curved portion of the prong 406, the average overall surface normal of the aperture may be used to determine the direction Fₙ that the fluid is directed from the tube.

The aperture 410 may be positioned at any position on the side of the prong 406 that allows the prong 406 to be placed in a cavity that directs the temperature-controlled fluid at a target area. If the prong 406 is curved to fit within one or more cavities of the subject, the orientation of the prong 406 within the nasal cavity 450 may be restricted. The aperture 410 may be positioned to take into account the restricted orientation of the prong 406. In some embodiments, the aperture 410 is positioned on the outside of a curvature of the prong 406. Accordingly, when inserted into a nasal cavity, the aperture may be positioned to be directed to the dorsal side of the subject. Fluid that is directed through the dorsal portion of the aperture 410 in the prong 406, when the prong is inserted into the nasal cavity 450, may be directed into one or more tissues of the oropharynx 234 and/or nasopharynx 232 of the nasal cavity. The term “dorsal” is used herein according to its broad and ordinary meaning and may refer to an anatomical orientation toward the posterior or back portion of the subject.

In some embodiments, the aperture 410 may be positioned on a side wall of the prong 406 close to the distal end 403 of the prong 406. This may allow for the temperature-controlled fluid 625 to be directed toward a wall of a cavity that is positioned at just less than the length of the prong relative to the connector hub 425 of the nasal cannula 400. In some embodiments, the aperture 410 is positioned on a side wall of the prong 406, a distance of between about 90% and about 100% of the distance between the proximal inlet end 402 and the distal end 403 of the prong. The position of the aperture 410, when referenced herein, may be determined based on a centroid of the aperture 410. In some embodiments, the aperture 410 is positioned on a side wall of the prong 406, a distance of between about 70% and about 80% of the distance between the proximal inlet end 402 and the distal end 403 of the prong 406. In some embodiments, the aperture 410 is positioned on a side wall of the prong 406, a distance between about 80% and about 90% of the distance between the proximal end and the distal end of the prong.

The connector hub 425 of the cannula can be configured to run perpendicular or axially to the prongs 406, and may serve as a supply tube connector and therefore may be considered a fluid supply connector hub. The connector hub 425 can comprise a short tube segment that serves as the central hub or base from which the nasal prongs 406 project, providing fluid communication between the prongs 406 and the fluid supply tubing. In some implementations, the connector hub 425 includes a swivel mechanism to allow the prongs 406 to rotate or adjust for a more comfortable fit within the nostrils, enhancing patient comfort and reducing the risk of the tubing twisting or kinking. The connector hub 425 can be configured to evenly distribute airflow from the supply tubing (not shown in FIG. 4) into the two nasal prongs 406. The connector hub 425 can further promote efficient delivery of cooling (or heating) fluid into both nostrils. The perpendicular orientation of the connector hub 425 relative to the prongs 406 can facilitate efficient management and direction of flow into the prongs 406. The connection of the connector hub 425 between the prongs 406 and the fluid supply tubing can provide desirable structural integrity for the nasal cannula 400 and for ensuring a secure pathway for fluid delivery.

In some embodiments, the aperture 410 is between about 1.5 inches and about 2.2 inches from the proximal inlet end 402 of the prong 406 along the longitudinal axis Aₚ of the prong 406. In some embodiments, the aperture 410 may be between about 1.2-2.0 inches from the proximal inlet end 402 of the prong 406 along the longitudinal axis Aₚ of the prong 406. In some embodiments, the aperture is between about 1.0 and about 1.2 inches from the proximal inlet end 402 of the prong 406 along the longitudinal axis Aₚ of the prong 406.

The aperture 410 may be configured to direct fluid flow through the prong 406 toward the roof and/or posterior wall 455 of the nasal cavity 450. By directing the fluid to the wall 455 of the nasal cavity, the fluid may physically interact with a specific target area on the top/back wall 455 before interacting with any other part of the body. Accordingly, the fluid that is passed through the prong 406 may be used to heat or cool the target area on the back wall 455. Various target areas in the posterior wall 455 may include a portion of nasopharynx 232 and oropharynx 234. The nasopharynx 232 is the posterior portion of the pharynx located behind the nose and above the soft palate 430. The oropharynx 234 is a lower posterior region of the pharynx between the soft palate 430 and epiglottis 435. In some embodiments, the prong 406 is configured to direct a temperature-controlled fluid against a wall of either or both of the nasopharynx 232 or oropharynx 234.

The specific target area within the nasopharynx 232 or oropharynx 234 may be selected based on the proximity to various arteries or other fluids that are distributed/circulated in the brain. For example, temperature-controlled fluid may be directed at an area of the nasopharynx 232 near or adjacent to the circle of willis. By directing fluid against tissues that are adjacent or near to fluids that are distributed to the brain, the disclosed prong 406 may control the temperature of the brain without undesirable side effects, such as systemic body cooling. Further, the shape of the prong 406 allows the temperature-controlled fluid to bypass various tissues, such as tissues within the nasal cavity from the nares 104 to the target area. Accordingly, heat exchange can be minimized in tissues outside of the target area, thereby reducing harmful effects on the subject from exposure to a temperature-controlled fluid.

FIG. 5 shows a cannula 500 with a prong 506 projecting from a fluid supply connector hub 525 according to an embodiment of the disclosed subject matter. Although only a single prong 506 is shown in FIG. 5, if should be understood that the cannula 500 may have two prongs, as shown and described in detail herein. The prong 506 can be attached to the connector hub 525, which may be configured as a connector hub configured to direct fluid from a lumen 505 of the connector hub 525 into a fluid channel/lumen of the prong 506. Fluid can be directed from the hub 525 into a proximal opening into the fluid channel of the prong 506 and out through the aperture 510. In various embodiments, the internal surface of the cannula may form a scooped outlet 512 that directs the fluid or gas out of the aperture 510 towards a target area. The scooped outlet 512 may have a radius of curvature that optimizes the release of the fluid or gas in a direction that is generally perpendicular to the axis A in the area of the side aperture 510. In some embodiments, the radius of curvature of the scooped outlet 512 may be greater than the curvature of the prong 506. In some embodiments, the radius of curvature of the scooped outlet 512 may be less than the curvature of the prong 506.

In some embodiments, the diameter of the aperture 510 is approximately larger than the diameter of the prong 506 as measured on a cross-sectional axis 530 that is perpendicular to a longitudinal axis A₁ of the prong 506. In some embodiments, the aperture 510 may be circular in shape, as shown in shape detail 550, and the diameter of the aperture 510 may be less than 0.15 inches, or about 0.15 inches, 0.2 inches, 0.25 inches, 0.35 inches, 0.4 inches, or greater. In other embodiments, the aperture 510 may be oval or capsule-shape, as shown in shape details 555 and 560. For rectangular implementations, the aperture 510 may have a length of between about 0.2-0.4 inches and/or a width of between about 0.15-0.25 inches. In other embodiments, the aperture 510 may have an irregular shape, such as a star shape, as shown in example shape detail 580. In such a configuration, the longest distance between two sides of the aperture of the shape detail 580 may be between about 0.2-0.4 inches.

With respect to the different example aperture shapes shown in the detail 501, the fluid egress direction/orientation/heading Fₙ may correlate to an average surface normal of the aperture 510 over the area thereof. Generally, the fluid egress direction Fₙ may be a direction pointing away from the prong 506, at a perpendicular angle θ₃ to the longitudinal axis A₁ in the area of the aperture 510. In some embodiments, the length of the prong 506 from the connector hub 525 to the distal end 520 is between about 1.5 inches and about 3.5 inches. In some embodiments, the prong 506 can be about 1, 2, 2.5, 3, 4.0, 5.0, 6.0, or 7.0 inches long.

The prong 506 may be curved to accommodate the shape of a nasal cavity. In some embodiments, a longitudinal axis A₁ of the prong 506 may be curved from the connector hub 525 to the distal end 520. The curvature of the prong 506 may be represented by an angle θ₂ between an angle/trajectory line 552 tangent to the longitudinal axis A₁ at the distal end 520 and an angle/trajectory line 545 tangent to the longitudinal axis A₁ at the connector hub 525. In some embodiments, the prong 506 may have a curve of about 5°, 10°, 15°, 20°, 25°, 30°, 35°, 40°, 45°, 50°, 60° or 75°. In some embodiments, the prong 506 may be formed of a material that is bendable or adjustable by the physician or the like to accommodate various shapes of a subject's nasal cavities. For example, the prong 506 may bend as it is inserted into a cavity.

Some embodiments of the prong 506 may be curved to adapt to the shape of a subject's nasal cavity. The curvature of the prong 506 may depend on the length of the prong 506 and the shape and size of the cavity of the subject. In some embodiments, the prong 506 of the cannula 500 may have a curvature of between about 0°, 5°, 10°, 15°, 20°, 25°, 30° or 35° per inch. In some implementations, the curvature of the prong 506 is designed to fit the nasal cavity's anatomy and direct airflow toward the nasopharynx and/or oropharynx. The curvature of the prong 506 can be defined by a radius of curvature of approximately 0.5-0.75″ inches. Such a configuration can allow the prong 506 to gently extend downward, minimizing discomfort while optimizing fluid delivery. The curvature of the prong 506 can effectively direct the flow of fluid towards the lower respiratory tract and maintain the prong's trajectory within the nasal passage, aiming the top-side aperture 510 towards the nasopharynx or oropharynx. The prong 506 can have a downward curvature orientation, as shown, which facilitates a precise extension towards the nasopharynx and/or oropharynx for efficient fluid delivery without causing undue user discomfort. The curvature of the prong 506 can follow an elliptical or parabolic path, with segments defined by different radii, such as transitioning from a radius of curvature of between 0.3-0.6 inches in a proximal portion 502 of the prong 506 near the fluid entry point at the connection to the connector hub 525, to a radius of curvature of between 0.6-0.9 inches in a distal portion 503 near the distal end 520.

As with any cannula prong disclosed herein, the prong 506 may be divided into, or considered to be divided into, a plurality of lengthwise segments or portions, including the proximal portion 502, which comprises the first one-third of the length of the prong 506 from the proximal base of the prong 506 running along the axis A1 of the prong. The proximal portion 502 of the prong 506 can be connected/adjacent to a medial portion of the prong 506 that extends between the proximal portion 502 of the prong 506 and a distal portion 503. The length of the distal portion 503 of the prong 506 can be the last/distal-most one-third lengthwise portion of the prong 506. All other embodiments of the prong 506 may be considered to include similar proximal, medial, and distal portions, which may be referred to using such terminology as a means for differentiating between the different lengthwise segments/portions of an elongated prong. As an example, if the prong 506 is 6 cm long, the proximal portion 502 can be considered to be the proximal-most 2-cm segment, the medial portion 504 can be considered to be the intermediate 2-cm segment, and the distal portion 503 can be considered to be the distal-most 2-cm segment. Similar portion distributions can apply for all other prong lengths, such as for prongs that are about 7-10 cm long, for example.

The curvature of the cannula 500 may change along the longitudinal axis A₁ from the connector hub 525 to the distal end 520. For example, the curvature of the prong 506 may vary from about 5° per inch at the connector hub 525 to about 20° per inch at the distal end 520. FIG. 5 shows an example angle/trajectory line 545 of the axis A₁ of the prong 506 at the proximal base of the prong 506, as well as an example angle/trajectory line 552 of the axis A₁ of the prong 506 at the distal end 520 of the prong 506. The angle θ₂ between the angle/trajectory line 545 and the angle/trajectory line 552 may advantageously be between 30-30°, between 35-40°, between 40-45°, between 45-50°, between 50-55°, between 55-60°, between 60-65°, between 65-70°, between 75-80°, between 80-85°, between 85-90°, or greater than 90°.

In some embodiments, the prong 506 may be tapered such that an outside diameter at a proximal portion of the prong 506 at the base connected to the connector hub 525 is larger than the outer diameter at the distal end 520 at the tip. In some embodiments, the outer diameter of the prong 506 at the base may be between about 0.1 or 0.3 inches and about 0.375 or 0.5 inches. In some embodiments, the outside diameter at the distal end 520 is between about 0.1 and 0.3 inches.

In various embodiments, the wall thickness of the prong 506 may be between about 0.01 or 0.02 inches and about 0.04 or 0.06 inches. The shape and size of the aperture 510 can vary. The aperture 510 may be a circle shape 550, an oval shape 555, a capsule shape 560, a rectangle shape 565 that is longer on a longitudinal axis A₁ than the cross-sectional axis 530, a triangle shape 570, a diamond shape 575 that is longer on the cross-sectional axis 530 than the longitudinal axis A₁, and a star-shape 580. The various shapes and sizes of the prong 506 may be selected based on the fluid mixture, the efficiency of heat distribution, the thermal conductivity of the fluid, and various other parameters.

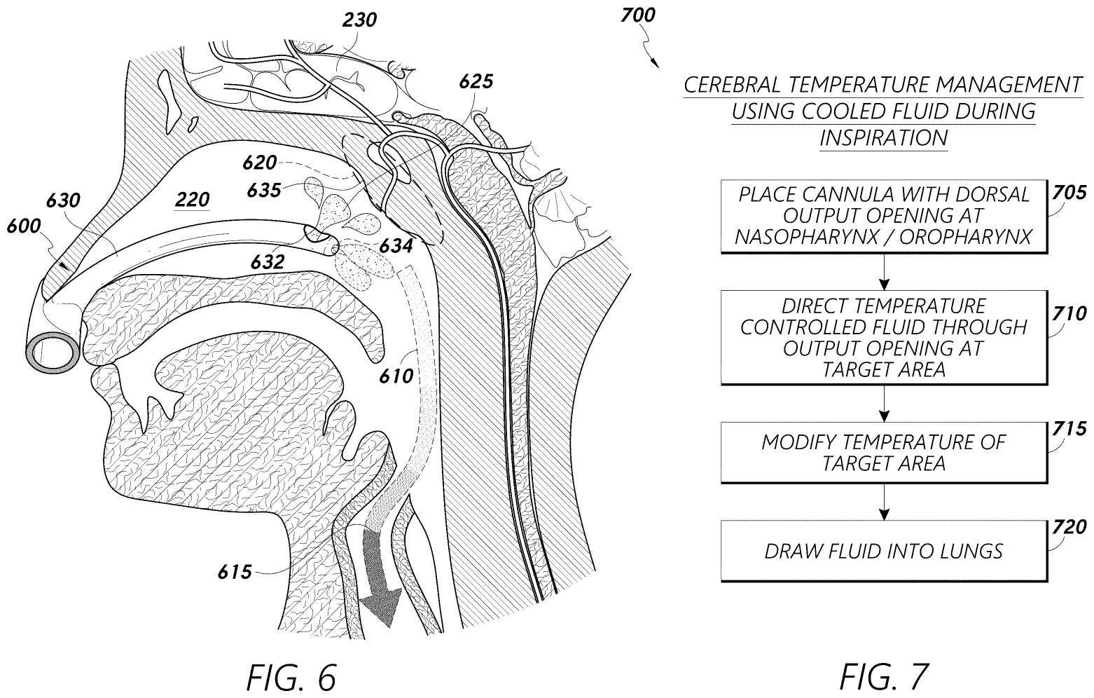

FIG. 6 is a side cross-sectional view of the nasal passageway/cavity 450 of a subject with arrows (610 and 615) that indicate inhalation into the respiratory system. Using the nasal cannula 600, a temperature-controlled fluid 625 may be directed through the nasal cavity 450 without interacting with bodily tissues until the temperature-controlled fluid 625 is ejected out of a prong 630 of the cannula 600 from a distal dorsal aperture 632 to a target area 620. For example, the shape of curvature and length of the prong 630 is such that the side aperture 632 faces the pharyngeal wall outside of the nasal passages/nostrils. Such features and processes can prevent or reduce the risk of the cooling effect or heat exchange with other body tissues until the fluid 625 reaches the target area 620. Once at the target area 620, the temperature-controlled fluid 625 exchanges heat with the target area 620 to raise or lower the temperature of the target area 620, which in turn may result in the cooling (or heating) of the temperature of blood vessels and/or other tissue in the vicinity of the target area 620/tissue. For example, the fluid 625 may cause cooling (or heating) of blood in the internal carotid arteries and/or the cavernous sinus and circle of willis, which may in-turn advantageously cool (or heat) the brain 230 with lesser or no cooling (or heating) of the trunk/core of the subject.

In some embodiments, the prong 630 is configured to deliver the temperature-controlled fluid 625 to the roof of the nasopharynx. The term “roof” is used herein according to its broad and ordinary meaning and may refer to the surface of a cavity (e.g., nasal cavity), where the surface is oriented toward a back or upper part of the cavity. In the side view of FIG. 6, the term “roof” refers to a surface of the nasopharynx that is adjacent to the brain, cerebral fluid, and various arteries that distribute blood to the brain. In some embodiments, the prong 630 may also include a second aperture 634 that is located at the terminal end of the prong. In the position as shown in FIG. 6, the distal aperture 634 may generally direct fluid expelled therefrom towards the larynx and ultimately into the lungs through respiration. It should be understood that the distal axial aperture 634 may be omitted in some embodiments. In some implementations, the second aperture 634 that is located at the terminal end of the prong 630 can be configured to help induce transpulmonary hypothermia with the cold fluid/gas mixture for cooling a subject rapidly. The second aperture 634 enables the prong 630 to release the cold fluid/gas mixture as shown by arrow 610 toward the lungs. The lungs have a sizeable capillary surface that can reduce the systemic temperature of a subject. The cold fluid/gas may contact the walls of the lungs, cooling the blood that enters the lungs for oxygenation. Freshly cooled blood can enter the heart and be circulated throughout the body, thereby reducing the temperature of a subject. In other embodiments, the fluid/gas may be heated/warmed and expelled through the distal aperture 634 towards the lungs to increase the systemic body temperature. In some embodiments, the prong 630 may be a nasopharyngeal tube, also known as a nasopharyngeal airway, which can comprise a flexible tube that is configured to be inserted through the nose and extends to the back of the throat or nasopharynx 620.

The temperature-controlled fluid 625 provides a cooling effect or heat exchange to the roof 635 at the target area 620. The target area 620 can comprise any pharyngeal wall or tissue of the pharynx. The term “pharynx” is used herein according to its broad and ordinary meaning, and may refer to an anatomical area that includes the nasopharynx and the oropharynx. For example, the pharynx may be considered to start at the back of the nose (e.g., nasopharynx) and extends to the oropharynx, and further to the laryngopharynx. The pharynx does not include the nasal passages or nostrils, which are the channels in the nose through which air enters and exits the respiratory system. Rather, the pharynx can be considered a part of the throat situated behind the mouth and nasal cavity, and divided into the nasopharynx (upper part of the pharynx, behind the nose), the oropharynx (middle part of the pharynx, behind the mouth), and the laryngopharynx (lower part of the pharynx). The pharynx generally extends to the esophagus and larynx. The wall at the back of the nasal cavity can be considered as a part of the pharynx wall, specifically as part of the nasopharyngeal wall. The posterior wall of the nasal cavity can be considered as the rear boundary of the nasopharynx. Both the nasopharynx and the oropharynx can be considered part of the pharyngeal wall areas. The terms “pharyngeal wall” and “pharyngeal wall area” are used herein to refer to the tissue wall structures of the pharynx, which can comprise mucosal lining, submucosal tissue, and muscular layers, and can further contribute to the functions of the pharynx in respiration, swallowing, and speech.

The arrow 610 indicates that the temperature-controlled fluid 625 approaches the lungs of the subject. As the temperature-controlled fluid mixes with the gasses from the body, heat is exchanged between the gasses and the fluid, tissues of the subject, and air within the respiratory tract. Accordingly, a temperature-controlled fluid 625 that exits the prong 630 from the aperture 632 at a temperature below the body temperature of the subject will become warmer as heat energy from the bodily tissues and air within the respiratory tract of the subject is transferred to the fluid 625. The temperature change of the fluid 625 is indicated by arrow 610 through shading as being warmer, as indicated by arrow 615 in FIG. 6. The normalization of the temperature as the fluid 625 approaches the lungs allows the subject to remain alert. One advantage provided by such configurations and processes may be that the subject may not have to be intubated or sedated while receiving a therapeutic cooling effect to reduce brain swelling after a brain trauma. During brain trauma treatment, it can be therapeutically beneficial to have the subject be alert in order to determine the responsiveness of the subject.

The curvature of the prong 630 can be considered to be in an inferior, posterior, and/or downward direction. That is, with respect to the illustrated dimensions, shape, and orientation of the prong 630 shown in FIG. 6, the curvature can deflect toward the back of the pharynx and downward, following a curve line that as extrapolated angles toward the larynx/throat.

FIG. 7 is a flow diagram of process 700 for cerebral temperature management using fluid during inspiration/inhalation, wherein such temperature management is performed using a nasal cannula in accordance with one or more embodiments of the present disclosure. The description of the process 700 can be understood with reference to FIG. 7, as well as FIG. 6, which shows certain device features and anatomy relevant to the process 700 of FIG. 7. As shown in FIG. 6, the prong 630 of the cannula 600 may be implemented to cool a target area 620 in a cavity, such as a nasal cavity, with minimal interaction with tissues until the treatment fluid reaches the target area 620.

At block 705, the process 700 can involve placing one or more prong(s) 630 of the nasal cannula 600 in a nasal cavity 450 of a subject, with a dorsal aperture 632 of the prong(s) 630 placed adjacent to a roof 635 surface/tissue at the target area 620. The prong(s) 630 may be placed so that the fluid egress aperture (e.g., output hole) 632 is positioned with a surface normal of the aperture directed toward the target area 620.

At block 710, the process 700 can involve directing temperature-controlled fluid 625 through the aperture 632 to the target area 620. In the example embodiment shown in FIG. 6, the cooled fluid 625 is directed to the nasopharynx/oropharynx area of the throat at target area 620. Specifically, the cooled fluid 625 is directed at the target area 620 adjacent to various arteries and brain fluid areas that may allow for rapid natural distribution of temperature changes to the rest of the brain 230.

At block 715, the process 700 can involve permitting the temperature-controlled fluid 625 to bring about a heat exchange at the target area 620 through the application of fluid 625 to the target tissue. For example, the fluid 625 can effectively cool the target area 620, wherein, after the heat exchange, the fluid 625 may become warmer. Accordingly, heat can be transferred from the target area 620 to the cool fluid 625 to increase the temperature of the fluid 625. In some embodiments, one or more systems of the body near or adjacent to the target area 620 may transfer heat to the fluid 625.

With the fluid 625 warmed by exchange with the target tissue/area 620, the warmed fluid can then pass to the rest of the respiratory tract, which can mitigate cooling in other portions of the respiratory tract. This may be beneficial in cases where temperature change is desired at the target area 620 but not the rest of the body. For example, it may be desired to cool the brain of a human or animal subject without cooling the rest of the body. In some implementations, it may be desired to increase the temperature of the subject, and thus, warm fluid 625 may be pumped into the subject's lungs and to the target area 620 to increase the systemic/core temperature of the subject.

At block 720, the process 700 can involve drawing at least a portion of the warmed fluid 625 into the lungs of the subject through inspiration. In some implementations, the fluid 625 brings about a heat exchange primarily with the target area 620 without significantly cooling other parts of the body. Accordingly, the fluid 625 that is delivered into the human body via the prong 630 does not significantly cool the lungs.

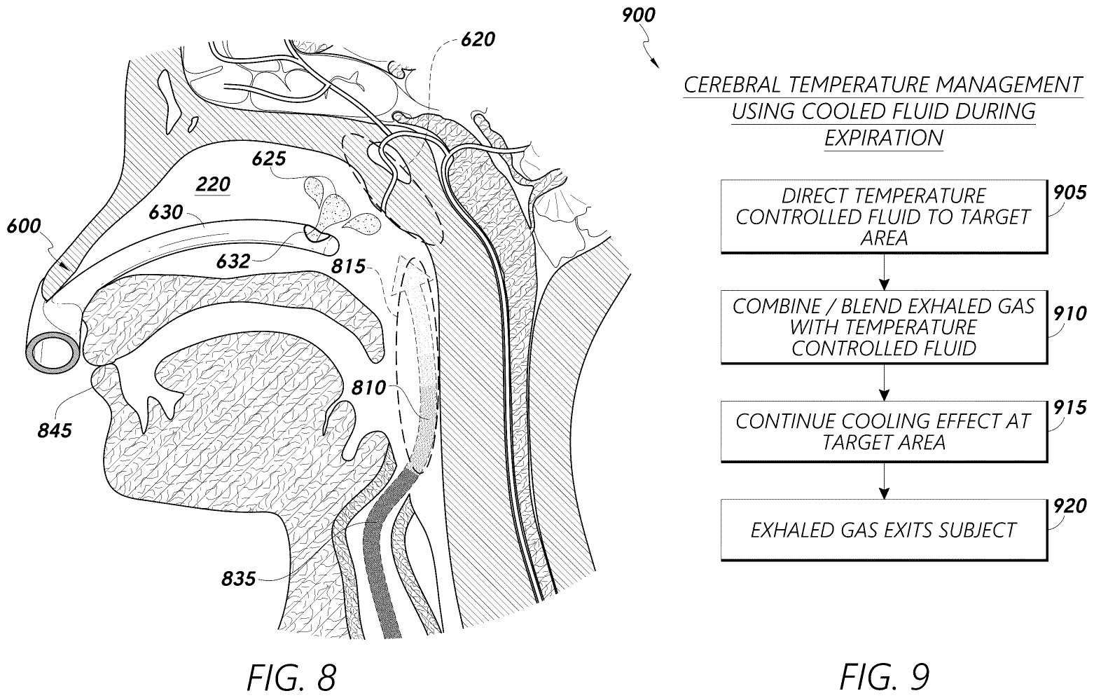

FIG. 8 is a side cross-sectional view of the respiratory system of a subject with arrows (810 and 835) that indicate exhalation of fluid. FIG. 9 shows a flow diagram of a process 900 relating to the imagery of FIG. 8. The process 900 and FIG. 8 relate to cerebral temperature management using cooled fluid during expiration/exhalation. The process 900 may occur after and/or in continuity with the process 700 described above with respect to FIGS. 6 and 7. At block 905, the process 900 can involve directing (e.g., continuing to direct) through the prong 630, temperature-controlled fluid 625 to the target area 620. Accordingly, the fluid 625 is directed through the prong 630 during inspiration and expiration without a change in flow rate or the cooling effect.

At block 910, the process 900 can involve combining/blending exhaled fluid/air at arrows 810 and 835 with the temperature-controlled fluid 625. Exhaled fluid (shown by arrows 810 and 835) may generally be exhaled from the lungs at approximately 32° C. This exhaled fluid (shown by arrows 810 and 835) can mix with the cooled fluid 625 expelled from the cannula 600, where the fluid passes from the larynx into the pharynx. The exhaled fluid at arrow 810 may cool as it passes through the oropharynx 815, where the cool fluid 625 delivered from the aperture 632 of the prong 630 mixes with the exhaled fluid at arrow 810. Generally, the gas/fluid exiting the lungs may be warmer than the fluid 625 expelled from the prong 630. Accordingly, heat energy will transfer from the fluid exiting the lungs (see arrow 810) to the fluid 625 expelled from the aperture 632, causing the fluid exiting the lungs to be cooled.

At block 915, the cooling effect continues as the mixed fluid reaches the target area 620. Accordingly, exhaled air (shown by arrows 810 and 835) that passes through the nasal cavity may propagate/increase a cooling effect from the cold fluid 625 expelled from the aperture 632.

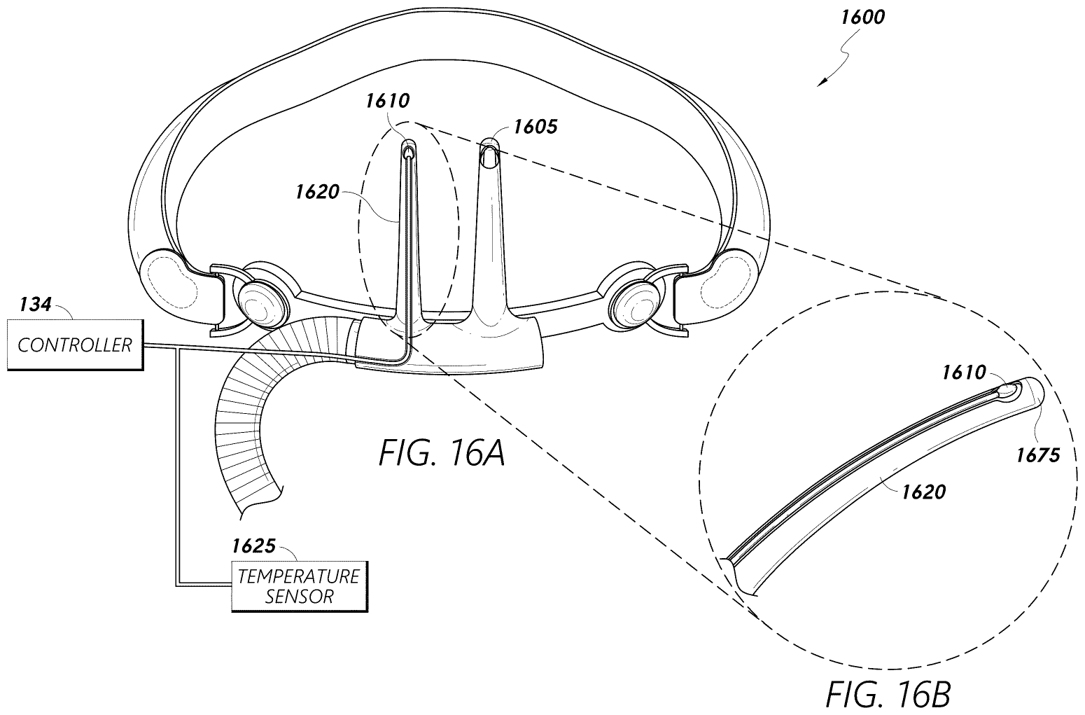

In some embodiments, the temperature of the fluid 625 propelled through the aperture 632 may be modified based on temperature measurements from one or more sensors placed within the respiratory tract (shown in FIGS. 16A and 16B). Measurements from a temperature sensor placed within the respiratory tract can be processed by the control circuitry 131 of a controller 134 to account for different temperatures of air exhaled from the lungs. For example, when cold fluid 625 is expelled through aperture 632, measurements from a temperature sensor in the pharynx or adjacent can be processed to account for periodic heating based on a respiration period. Cooling temperature of the working fluid can be cyclically adjusted to account for respiration conditions to produce an even cooling temperature across the respiratory cycle.

The respiration period may indicate various physiological responses in the subject to treatment with the nasal cannula treatment. The respiration period may also indicate various physiological responses to trauma or other physiological damage experienced by the subject. In an example embodiment, a controller (e.g., controller 134 in FIG. 2) may be configured to increase the temperature of the temperature-controlled fluid 625 in response to an increased respiration rate to account for possible respiration rate disruption of the cooling effect of fluid 625. At block 920, the process 900 may involve exhaled fluid/air exiting the subject. In some embodiments, the exhaled fluid may exit the subject through the mouth or nostril(s). Accordingly, the subject may continue ordinary respiration during the cerebral temperature management process.

In various embodiments, the cannula 600 may be inserted into both nares of the subject. If fluid exhaled from the lungs cannot exit the subject through the nasal cavity 450 due to partial obstructions caused by the presence of the prong(s) 630, the exhaled fluid may exit the subject through the oral cavity 845. In some embodiments, the cannula 600 may be inserted into only one nare, thus leaving the other nare available for fluid to exit the subject.

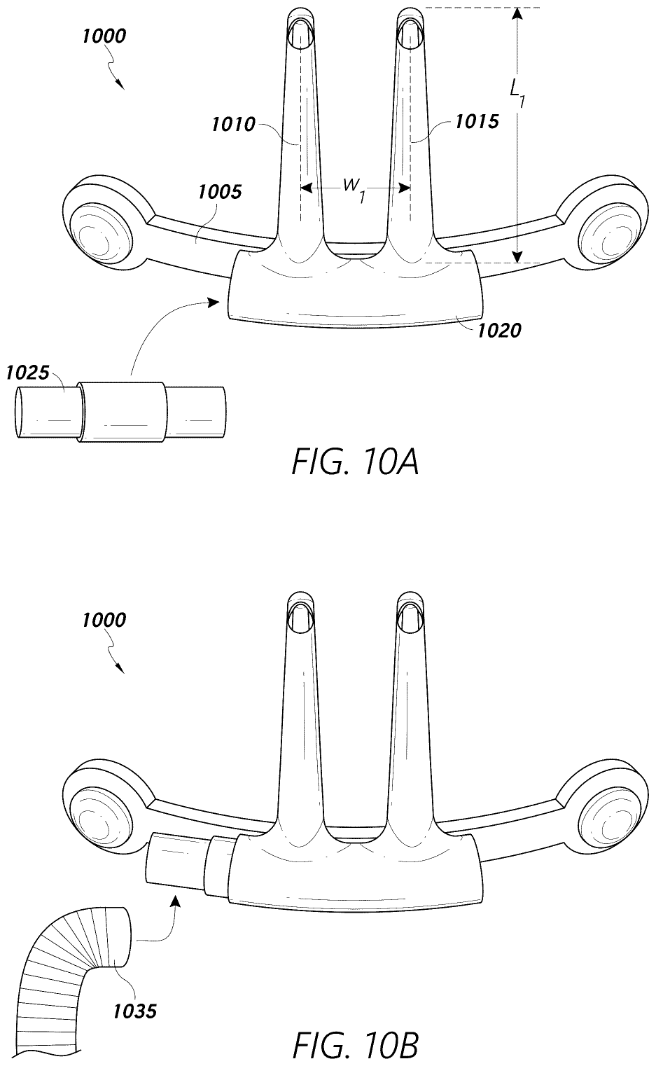

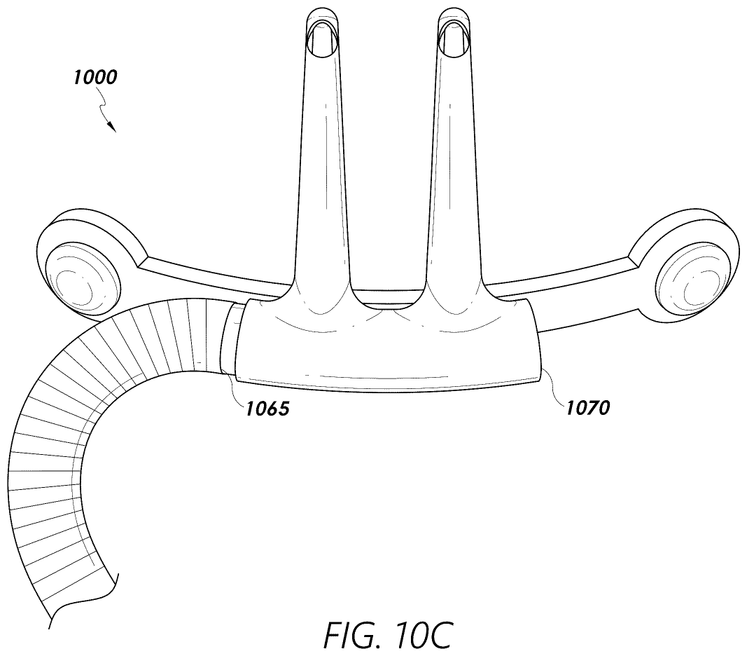

FIG. 10A shows a cannula 1000 that is configured to be inserted into the nasal cavity of a subject. In the example embodiment shown in FIG. 10A, the cannula 1000 includes various components such as a support structure 1005, a pair of nasal prongs 1010 and 1015, a fluid supply connector hub 1020, and a tube connector 1025. The cannula 1000 is designed to deliver a fluid to a target area of the nasal cavity of a subject, as described in detail herein.

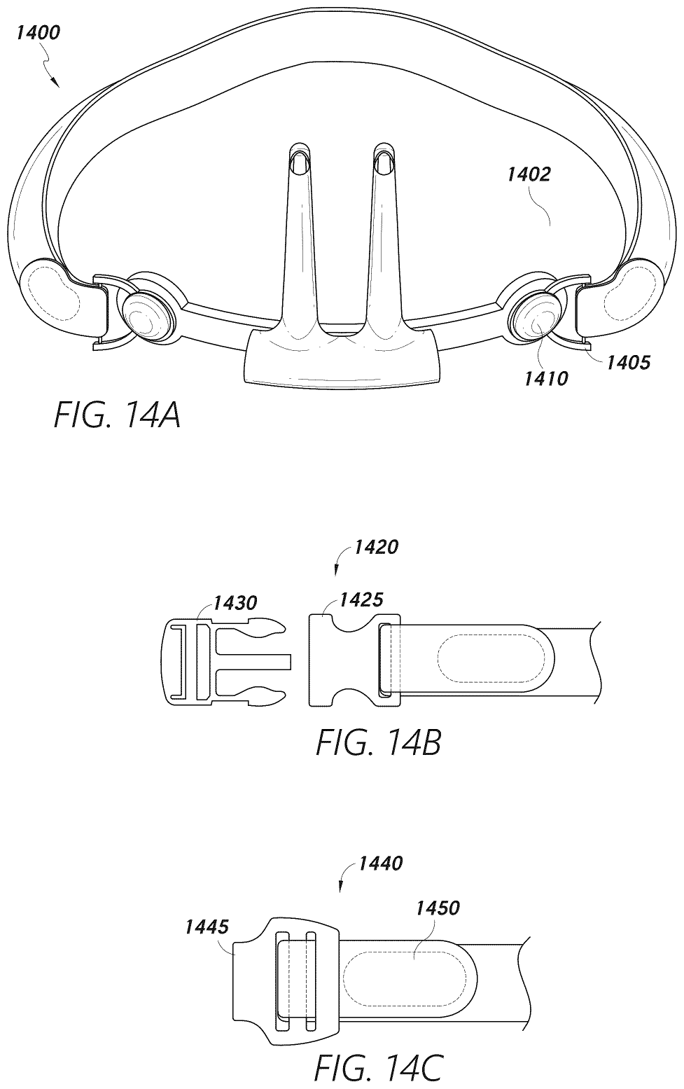

The cannula 1000 includes the tube connector 1025, through which the fluid can be supplied to the pair of nasal prongs 1010 and 1015 by connecting the tube connector 1025 to an external fluid source that causes fluid to flow through the tube connector 1025. The support structure 1005 is configured to support the connector hub 1020 and enable the subject to wear the cannula 1000 by using fasteners (shown in greater detail in FIGS. 14A, 14B, and 14C). The connector hub 1020 can be configured to receive the pair of nasal prongs 1010 and 1015. Further, one end of the connector hub 1020 can be connectable to the tube connector 1025, and the other end of the connector hub 1020 can be sealed. The pair of nasal prongs 1010 and 1015 can be the elongated prongs that are insertable into the subject's nostrils. The pair of nasal prongs 1010 and 1015 is configured to administer temperature-modifying fluid to the subject by positioning and fitting the cannula 1000 into the subject's nostrils.

FIG. 10B shows the embodiment from FIG. 10A, displaying fluid tubing 1035 that can be connected using the tube connector 1025. In the example embodiment shown in FIG. 10B, the cannula 1000 may have the support structure 1005, the pair of nasal prongs 1010 and 1015, the connector hub 1020, the tube connector 1025, and the fluid tubing 1035. One end of the fluid tubing 1035 is connected to the tube connector 1025, and the other end of the fluid tubing is connectable to an external fluid source, which supplies the fluid to the cannula 1000.

FIG. 10C shows the embodiments from FIGS. 10A and 10B with the fluid tubing 1035 connected to the cannula 1000. The cannula 1000 may include the support structure 1005, the pair of nasal prongs 1010 and 1015, the connector hub 1020, the tube connector 1025 (which is covered), and the fluid tubing 1035. One end of the fluid tubing 1035 is connected to the connector hub 1020 through the tube connector 1025 at a first end 1065 of the connector hub 1020, and the other end of the fluid tubing 1035 is left open and is connectable to a fluid source.

In some embodiments, the fluid tubing 1035 may be connected to the second end 1070 of the connector hub 1020. Two units of fluid tubing 1035, each connected to a separate fluid source, may be connected to the first end 1065 and second end, respectively. In an example embodiment, a first fluid tubing 1035 is connected to the first end 1065 of the connector hub 1020 and a second fluid tubing 1035 is connected to the second end 1070 of the connector hub 1020 where the first fluid tubing and second tubing are connected to different fluid sources. The fluid sources from the first fluid tubing 1035 and the second fluid tubing 1035 may be switched on and off to quickly change one or more properties of the fluid being directed through the cannula 1000.

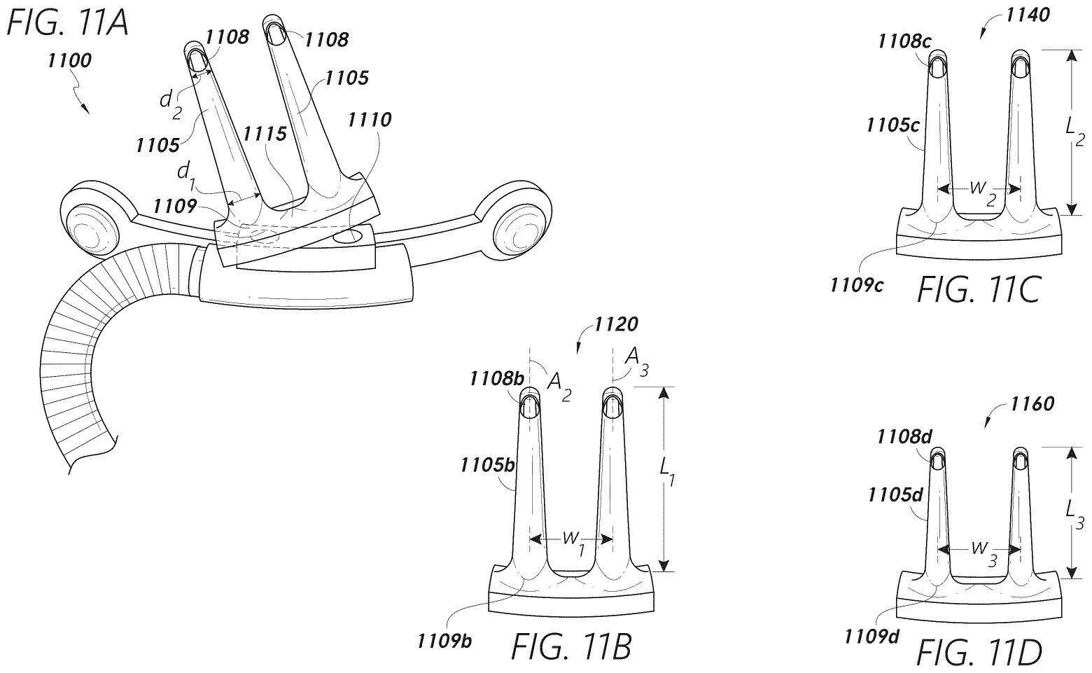

FIG. 11A shows a cannula 1100 with a removable pair of prongs 1105. The prongs 1105 are removably connectable to a connector hub 1110. The prongs 1105 can be removable, and friction fit onto the cannula 1100 based on the size of the nasal cavity of the subject. The cannula 1100 is modifiable to accommodate subjects of different sizes. The prongs 1105 are connected via a base 1115. The base 1115 is friction fit onto the connector hub 1110.

Each prong of the removable pair of prongs 1105 may include a proximal portion 1109 connected to the base 1115 and a free distal portion 1108 at a tip of each of the removable pair of prongs 1105. The prongs 1105 may be tapered, whereby an outside diameter d1 at the proximal portion 1109 is different from an outside diameter d2 of the prongs 1105 at a distal portion 1108. For example, an outside diameter d1 at the proximal portion 1109 may be larger than the outside diameter d2 of a distal portion 1108 such that the outside diameter decreases along the longitudinal axis from the proximal portion 1109 to the distal portion 1108.

FIG. 11B shows an alternative embodiment of a removable pair of prongs 1120. If the subject has a large nasal cavity, the removable pair of prongs 1105b may be of a larger size. In some embodiments, the pair of prongs 1105b may have a length L₁ between about 3.0-4.5 inches. The outside diameter at a proximal portion 1109b may be between about 0.4-0.5 inches. The outside diameter at the distal position 1108b of the prongs 1120 may be between about 0.25-0.35 inches. In various embodiments, the distance (or width w₁) between the pair of prongs 1105b is adapted to the inter-nostril distance of the subject. The inter-prong distance w₁, also known as the “prong-separation distance,” refers to the linear distance between the center axes of the prongs 1105. In various examples, the inter-prong distance may be measured in a straight line between and perpendicular to the adjacent prong axes (e.g., axis A₂ and axis A₃). The inter-prong distances of embodiments of the present disclosure may be between about 0.4-0.7 inches for embodiments designed for adult human subjects. The distance w1 between the pair of prongs 1105b may be between about 0.7-1.0 inches in some embodiments.

In any of the embodiments disclosed herein, the prong length L₁ of a nasal cannula prongs can advantageously be at least twice as long as the prong-to-prong distance w₁ (i.e., “inter-prong distance” or “prong-separation distance”). In some embodiments, the prong length L₁ is three (3×), four (4×), five (5×), six (6×), second (7×), eight (8×), nine (9×), ten (10×), or more times the prong-separation width w₁ of the prongs 1105. Such relatively long prong lengths can advantageously allow for access to anatomy deep within a target cavity/passage by a distal portion of the prong(s), such as for directly cooling the oropharynx or nasopharynx through the nasal cavity.

FIG. 11C is another illustration 1140 of a removable pair of prongs 1105c. If the subject has a medium-sized nasal cavity, the removable pair of prongs 1105c implemented may be smaller than the size of the prongs in FIGS. 11A and 11B. In some embodiments, the pair of prongs 1105c may have a length L₂ between about 2-3.0 inches. The outside diameter at a proximal position 1109c of the prongs 1105c may be between about 0.3-0.4 inches. The outside diameter at a distal position 1108c of the pair of prongs 1105c may be between about 0.18-0.25 inches. In some embodiments, a medium-sized pair of prongs 1140 may be between about 60% and about 80% of the size of the larger pair of prongs 1120. In some embodiments, the medium-sized pair of prongs 1105c may be between about 50% to about 60% of the size of the larger pair of prongs 1105b in length and/or outside diameter. The distance w₂ between the pair of prongs 1105c may be between about 0.5 inches and about 0.7 inches.

FIG. 11D is another illustration 1160 of a removable pair of prongs 1105d. If the subject has a small-sized nasal cavity, the removable pair of prongs 1105d implemented might be of a smaller size compared to the large-sized pair of prongs 1105b and a medium-sized pair of prongs 1105c.

In some embodiments, the pair of prongs 1105d may have a length L3 of between about 0.5 inches and about 2.0 inches. In some embodiments, the pair of prongs 1105d may have a length of between about 1.0-2.0 inches. The outside diameter at a proximal position 1109d of the prongs 1105d may be between about 0.1-0.3 inches. The outside diameter at a distal position 1108d of the pair of prongs 1105d may be between about 0.02-0.1 inches. In some embodiments, the outside diameter at a proximal position 1109d of the pair prongs 1105d may be between about 0.05-0.1 inches. In some embodiments, the pair of prongs 1105d may be configured to be even smaller in size based on the needs of the subject, such as for an infant subject. The distance w₃ between the pair of prongs 1105c may be between about 0.2-0.5 inches.

In some embodiments, the smaller size pair of prongs 1105d may be between about 10% to 50% the size of the larger sized pair of prongs 1105b in length and/or outside diameter. In some embodiments, the smaller-sized care of prongs 1105d may be between about 2% to 10% of the size of the larger-sized pair of prongs 1105b in length and/or outside diameter.

FIG. 12A shows a cannula 1200 with a pair of individually removable prongs 1205 and 1210. The individual removable prongs 1205 and 1210 are connected to a connector hub/base 1215 of the cannula 1200. The prongs 1205 and 1210 can be individually removable and inserted into the cannula 1200 based on the size of the subject. The height of the prong 1205 may be 16, 17, 18, 19, 20, 25, or 30 times the width of the prong 1205.

In some embodiments, the removable individual prong feature of the cannula 1200 may be combined with the base 1215 feature of the cannula 1200. Accordingly, a cannula 1200 may comprise a removable base with one or more individual removable prongs 1205. Both the base 1215 and individual removable prongs 1205 and 1210 may be swapped out based on the medical practitioner's and subject's needs.

FIGS. 12B, 12C, and 12D show prongs 1220, 1240, and 1260, respectively. Prong 1220 varies in both thickness and length compared to prong 1205. The height d₄ of prong 1220 may be 11, 12, 13, 16, or 15 times the width w₄ of the prong 1220. The height d₅ of prong 1240 may be 2, 3, 4, 5, 6, 7, 8, 9, or 10 times the width w₅ of the prong 1240. The height do of prong 1260 may be 16, 17, 18, 19, or 20 times the width w₆ of prong 1260.

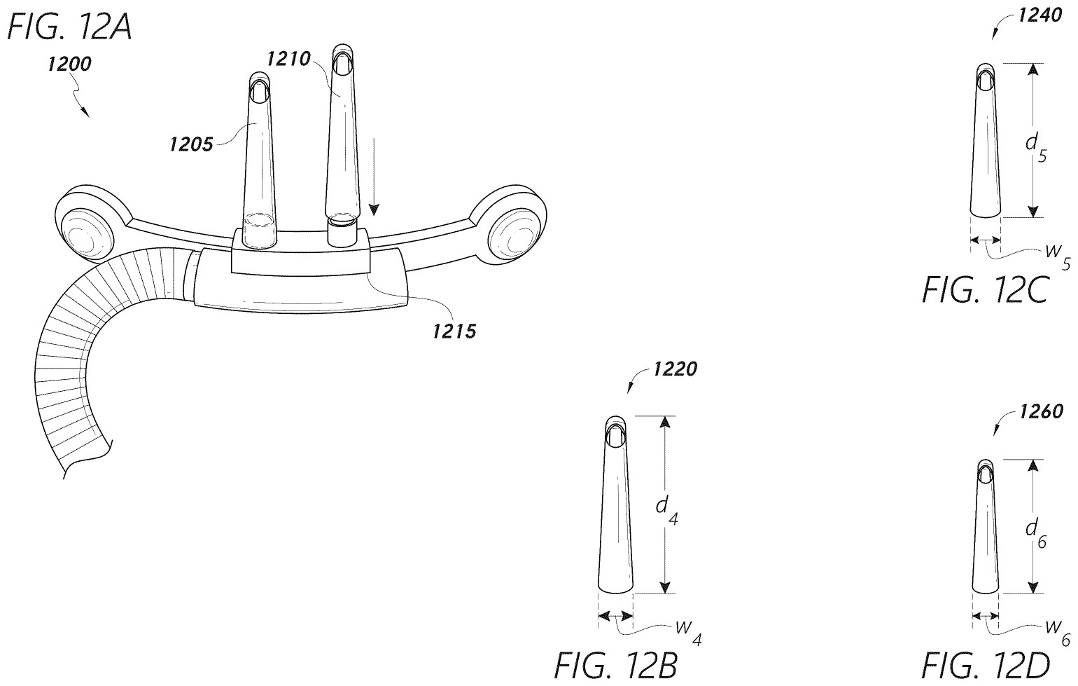

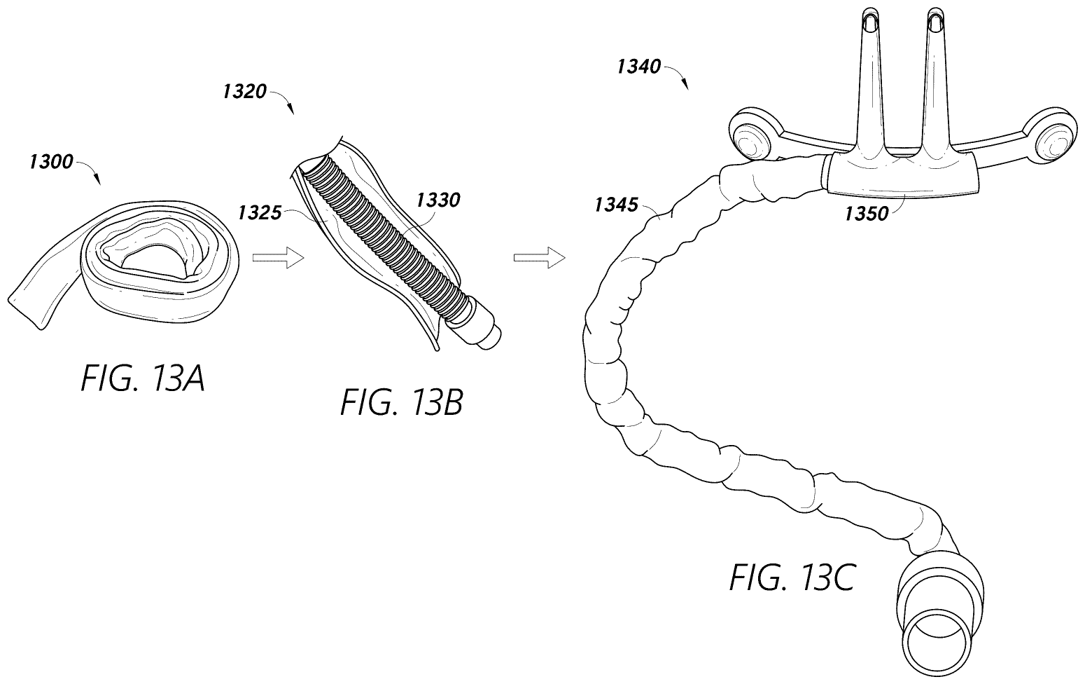

FIG. 13A shows an insulating sleeve 1300 configured to surround a fluid supply tube 1330. The insulating sleeve 1300 is a protective covering designed to provide thermal insulation to the fluid supply tube 1330. FIG. 13B shows an illustration 1320 of the fluid supply tube partially covered by the insulating sleeve 1325. The insulating sleeve 1325 is partially covered by the fluid supply tube 1330 to provide thermal insulation.

FIG. 13C shows an illustration 1340 of the fluid-supplying tube covered 1330 by the insulating sleeve 1345 connected to the cannula 1350 with prongs that are configured to deliver fluid into the nasal cavity of a subject. The fluid supply tube 1330 may be completely covered by the insulating sleeve 1345.

FIG. 14A is an illustration of a cannula 1400 with a fastening device 1402 in accordance with an embodiment. The fastening device 1402 is a component that is designed to secure or fasten the cannula 1400. In some embodiments, the fastening device 1402 is in the form of adjustable head straps. The head straps that go around the head of the subject may secure the cannula 1400 to the subject's head. Further, the head straps are adjustable, which allows them to be modified to fit different head sizes, providing a versatile and customizable solution.

The fastening device shown in FIG. 14A comprises a hook 1410 and an anchor 1405 fastener. A hook 1410 of the fastener hooks around and anchor 1405 to fasten and secure the cannula 1400 to the subject's head. In some embodiments, the cannula 1400 may comprise one or more fastening devices.

FIG. 14B shows another embodiment of a fastening device 1420. The fastening device 1420 comprises a buckle-type fastener. The buckle fastener comprises a female portion 1425 that is configured to receive a male portion 1430 to secure and fasten the cannula 1400 to the subject's head.

FIG. 14C shows another embodiment of a fastening device 1440. The fastening device 1440 comprises a strap 1450 and adapter 1445. The strap 1450 may be fed through the adapter 1445 to secure and fasten the cannula 1400 to the subject's head. The fastening device 1440 comprises head straps that include elastic material, thereby providing flexibility and accommodating various head sizes comfortably.

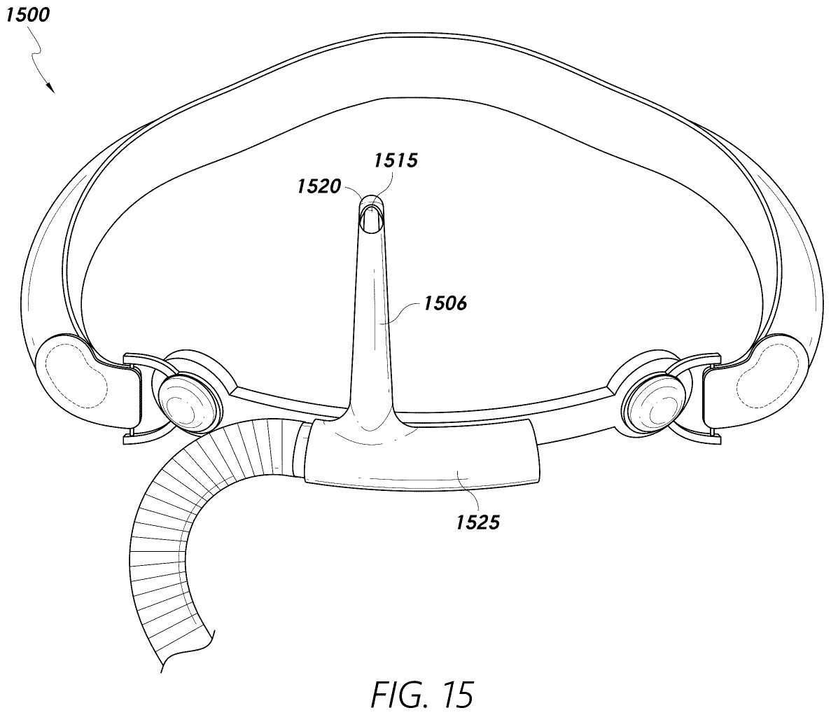

FIG. 15 shows a cannula 1500 with a single prong 1506 that is configured to be inserted into a nasal cavity. The cannula 1500 is an alternative to an embodiment with a pair of prongs configured to be inserted into two nares of a nasal cavity simultaneously. In some embodiments, the prong 1506 may be sized to allow fluid passage through a connector hub 1525 and the prong 1506 and out an aperture 1515 on a side of the prong 1506 near a distal end 1520 to cool (or heat) the nasal oropharynx or nasopharynx without the fluid interacting with tissues in between the nare and the nasal oropharynx/nasopharynx.

Any of the embodiments disclosed herein may be implemented as a single-prong cannula. With this design, the unused nare access may be used to facilitate the insertion of an additional device into the nasal cavity, such as a nasogastric tube, a suction tube, monitoring equipment, or performing a medical procedure on the patient with a medical device. In addition, the open nare may also act as a pressure relief in the event the flow rate of the delivered fluid exceeds the maximum inhalation volume of the patient.

FIG. 16A shows a cannula 1600 having a first prong 1605 configured to deliver fluid to a nasal cavity and a second prong 1620 configured to measure a temperature in the nasal cavity. The second prong 1620 can have a thermocouple probe that may be used to monitor the temperature of the nasopharynx/oropharynx of the subject. The first prong 1605 and second prong 1620 can have a common length. The term “common length,” as used herein, can refer to two or more lengths that are within 10% of one another relative to the longest length of the two or more lengths. In some implementations, either of the prongs 1605, 1620 may be longer than the other by an amount of more than 10%, such that the prongs 1605, 1620 are not considered to have a common length.

As an example, the first prong 1605 may deliver a temperature-controlled fluid to the subject at a target area, as described in detail herein, while the second prong 1620 can comprise a temperature probe 1610 that may receive one or more measurements at or near the target area. In some embodiments, one or more variables related to a flow and/or temperature settings/levels of fluid passed through the first prong 1605 may be adjusted based on measurements made by the second prong 1620.

The design of the first prong 1605 and the second prong 1620 can be asymmetrical, with a smaller outer circumference prong housing the temperature sensor/probe 1610. The temperature probe 1610 can be used for accurate core temperature measurement. The temperature probe can be positioned and applied in close contact with nasopharyngeal or oropharyngeal mucosa to reflect the blood temperature.

In some embodiments, the second prong 1620, which serves as a nasopharyngeal temperature probe, may be configured to measure the target area or some point outside of the target area a distance away from the distal aperture of the first prong 1605. For example, the second prong 1620 may be shaped to extend further down the pharynx than the first prong 1605 to measure the temperature of the fluid as it travels further down the respiratory tract. In another example, the second prong 1620 may be shaped to make contact with tissue in the target area to make physiological measurements directly instead of measuring the temperature of the fluid being delivered through the first prong 1605 and/or other ambient media.

FIG. 16B is a zoomed-in view of 1650 of the second prong 1620 illustrated in FIG. 16A. In some embodiments, the second prong 1620 may comprise a smooth curved shape configured to be inserted into a cavity such as a nasal cavity. The second prong 1620 may have the temperature probe 1610 on or within the side wall of the second prong 1620. The temperature probe 1610 is positioned on the side of the second prong 1620 close to the distal end 1675. For instance, the temperature probe 1610 may be positioned between about 0.1-0.5 inches from the distal end 1675. In some embodiments, the temperature probe 1610 may be positioned between about 0.6-1.0 inches of the distal end 1675. Accordingly, the temperature probe 1610 may be positioned to make contact with a wall of the cavity. In some embodiments where the temperature probe 1610 is inserted into various other portions of the subject besides the nasal cavity, the sensor may be positioned to make contact with one or more tissues of the target area.

In the example embodiment of the second prong 1620 shown in FIG. 16B, the temperature probe 1610 can be a thermocouple, which may be configured to detect thermal energy in accordance with the Seebeck effect, which converts a voltage difference between two positions of a metal to a temperature difference between the two positions. Although only one of the prongs in FIG. 16A is shown and described as having a temperature sensor, it should be understood that both the first prong 1605 and second prong 1620 may be temperature sensors associated therewith. Furthermore, any prong having an associated temperature sensor may further include a cooling (or heating) fluid channel and fluid egress aperture for temperature management, as described herein. For example, temperature management fluid may flow within the fluid channel with the temperature sensor(s) disposed on an outside of the prong and/or channel, or the sensor may be disposed within the fluid channel such that the fluid flows around/aside the sensor. In various embodiments, the prong with a temperature sensor may be void of a fluid channel. The sensor(s) may be affected by the flow in the prongs. In other embodiments, one or more sensors 1625 may be placed at any other location of the system. The sensor(s) 1625 can be located at various locations of the subject being treated. Example locations of the sensor(s) 1625 may include one or more sensors configured for placement in an underarm, rectal, or mouth area, or in a position/area touching the nasal or sinus cavity or the pharyngeal wall/tissue. The signals generated by sensor(s) 1625 may be provided to the controller 134 (see FIG. 2), or any other control circuitry of the system, and the controller/circuitry 134 may change the temperature or flow rate of the fluid based on the measurements received from sensor(s) 1625. For example, measurements from sensor(s) 1625 positioned outside of the nasal cannula 1600 can be used as a proxy from which pharyngeal or brain temperature can be determined/inferred.

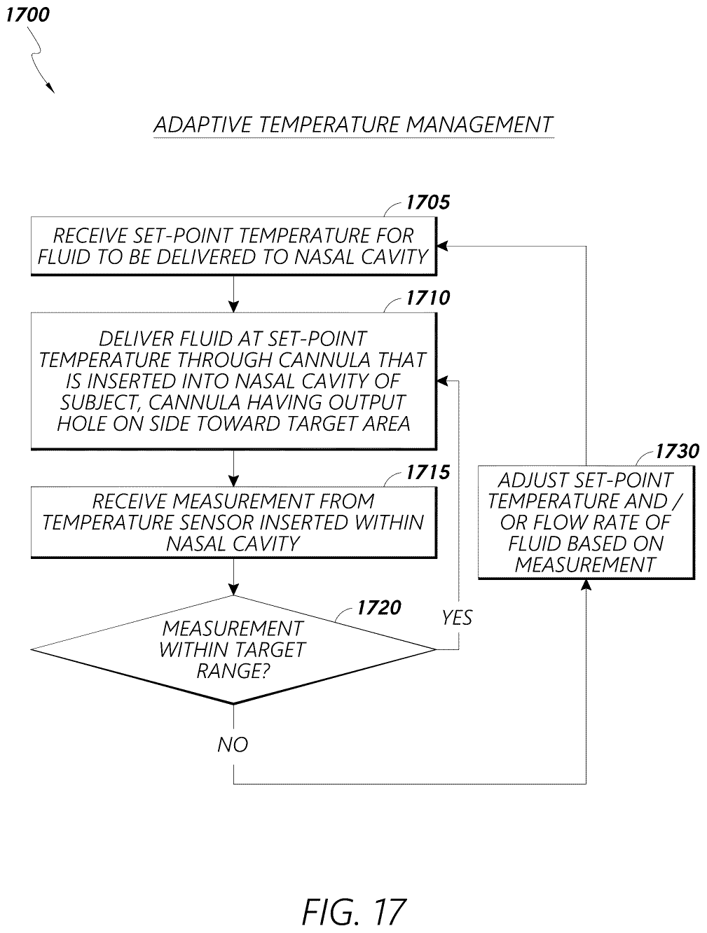

FIG. 17 is a flow diagram of a process 1700 for delivering fluid to a subject using the systems disclosed above. At block 1705, control circuitry 131 of a temperature management system 100 (e.g., control circuitry of controller 134 in FIG. 2, embodied in one or more devices/systems) may receive a set point temperature for fluid to be delivered to a subject. The temperature may be the desired temperature of the fluid to be delivered to the subject. For example, a set point temperature of 10° C., 15° C., or 20° C. may be set for a fluid that is to be delivered to a target area in the oropharynx or the nasopharynx to cool the brain. In various embodiments, the set point temperature should not be allowed to be less than 0° C.

In an alternative embodiment, the set point temperature may be the desired temperature of the target area or other position in the subject. For example, the set point temperature may be the desired temperature of anatomy (e.g., tissue, fluid) disposed in proximity to a position of at least a portion of the brain. A controller may process the set point temperature, which represents a desired temperature for a target area in a subject, and determine an ideal temperature for the fluid to be delivered to the subject.

At block 1710, the controller 134 may deliver fluid at the set point temperature through a cannula that is inserted into the nasal cavity of a subject where a prong of the cannula has an aperture on a side wall thereof, as described in detail herein. The aperture may be directed toward a target area within the oropharynx and/or nasopharynx of the subject. In some embodiments, the fluid is delivered via any prong that penetrates the cavity and delivers the fluid without allowing the fluid to interact directly with the tissues of the subject before being expelled through the aperture.

At block 1715, the controller 134 may receive a measurement from a temperature probe that is inserted within the nasal cavity of the subject. In some embodiments, the temperature probe may be positioned in other cavities or other portions of the subject. In some embodiments, the temperature probe may be replaced or supplemented by another type of sensor, such as a blood pressure sensor, an oxygen level sensor, a brain oxygen sensor, a respiration sensor, an inflammation sensor, or any other physiological sensor that may be used in a medical setting.

At block 1720, the controller 134 determines whether the measurement is within a target range and/or reaches a predetermined threshold level. For example, a range of measurements may be between about 20-33° C. for some use cases. For example, when the nasal cannula 400 is intended to heat the target area or the subject as a whole, the setpoint temperature range may be about 33-38° C. If the temperature measurement is within the target range, or otherwise meets the compared temperature state/threshold, the process 1700 may continue to block 1710 and deliver fluid at the set point temperature. If the measurement is outside of the target range, the process 1700 may proceed to block 1730 to make adjustments.

At block 1730, the controller 134 may adjust a set point temperature or the flow rate of the fluid based on the temperature measurement. For example, a set point temperature of the fluid may be adjusted upward or downward based on the measurement. A measurement of the target area 112 that is higher than expected, the controller 134 (see FIG. 2) may adjust the set point temperature downward and/or increase the flow rate of cooler fluid. Likewise, a low target area temperature measurement may result in an upward adjustment of the set point temperature or a reduction in the flowrate of warmer fluid.

In addition to the adjustment of the set point temperature, the flow rate of the fluid may be adjusted. For example, a temperature of the measurement of the target area that is higher than expected may result in an increase in flow rate. In some embodiments, a range of flow rates that may be delivered for fluid by the disclosed subject matter may be between about 1 liter per minute (lpm) and 40 lpm. Likewise, measurements in the target area 112 that are too low may result in a decrease in flow rate.

In some embodiments, the adjustment to the flow rate or set point temperature may be performed by an automated process. For example, a measurement may be received by a controller 134 that automatically adjusts a set point temperature or fluid flow rate of the temperature-controlled fluid that is delivered to the aperture of the cannula 102. In some embodiments, the controller may adjust a mixture of the fluid as well. For example, the controller 134 may increase or decrease the oxygen percentage of the fluid being delivered to the human or animal subject.

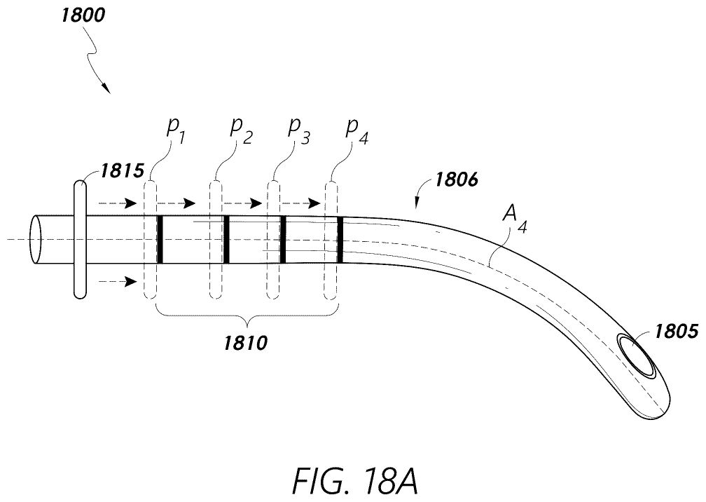

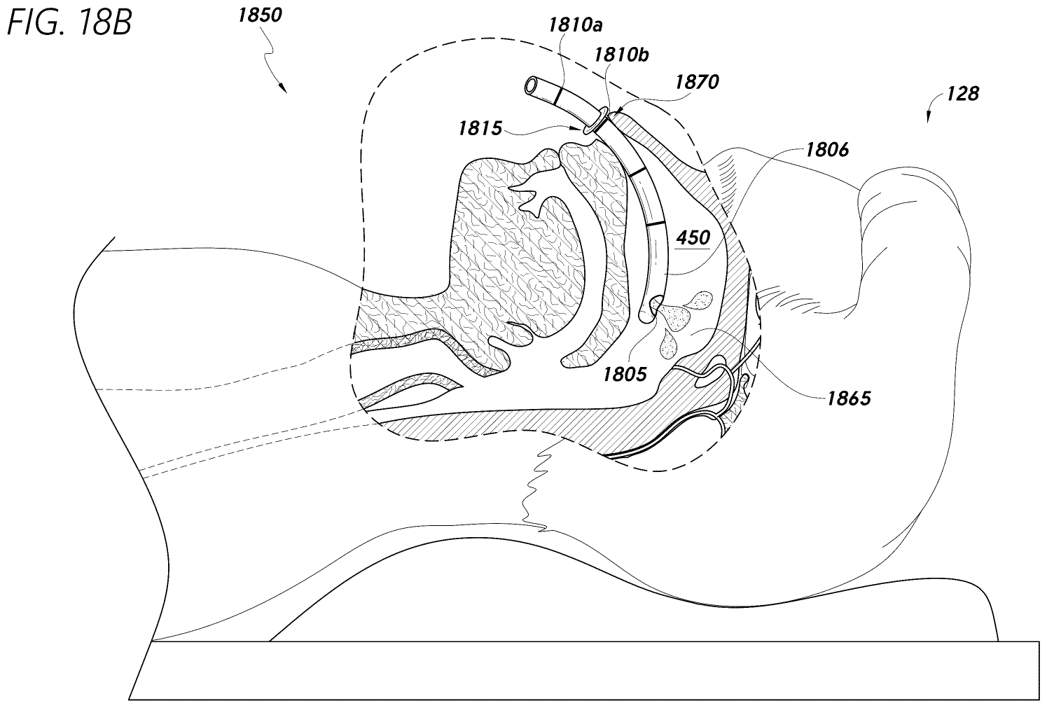

FIG. 18A is an illustration of an embodiment of a cannula 1800 comprising one or more prongs 1806 with depth markings 1810 implemented thereon. FIG. 18B is an illustration of the embodiment of the cannula 1800 shown in FIG. 18A as it is inserted into the nasal cavity 220 of a subject 128. It is anticipated that various subjects will have differently shaped nasal cavities that require the prong 1806 to be inserted at various depths depending on the size of the subject. For example, the example embodiments shown in FIGS. 11A-12D illustrate various interchangeable prongs of different sizes and shapes. In addition to embodiments that allow for elongated shapes to be interchanged for different sizes, prong insertion depth customization may further be implemented using nasal cannulas that have prong depth markings and/or adjustable proximal stoppers to aid in setting an insertion distance for a nasal cannula. For example, the prong 1806 may be inserted at different depths as indicated by the depth marking(s) 1810 and/or set by the proximal stopper 1815 to accommodate different sizes of nasal cavities.

The depth markings 1810 may provide visual indicators to indicate the position of the prong 1806 as it enters the cavity of the subject. Accordingly, the prong 1806 may be inserted up to the depth marking 1810 to confirm that the aperture 1805 is at the target area, the distance to which may be measured or determined pre-operatively. The depth markings may comprise rings, lines, pointers, or other indicators implemented on the shaft of the prong 1806, wherein such indicators may be implemented as recesses, projections, printed/painted markings, or the like.

In some embodiments, the cannula 1800 may include a proximal stopper 1815, such as a flange, lip, collar, or other structure configured to limit the insertion depth of the prong 1806 at the axial position of the proximal stopper 1815. For example, once a distance from the cavity aperture to the target area is determined, a depth marking 1810 may be selected, and the proximal stopper 1815 may be moved along the longitudinal axis A₄ to positions such as p₁, p₂, p₃, and p₄ to the appropriate depth marking 1810.

As shown in illustration 1850 in FIG. 18B, the prong 1806 may be inserted to align the aperture 1805 of the prong 1806 with a target area of the subject. An example of the target area may be the oropharynx or nasopharynx in the throat of the subject. Depth markings 1810 on the prong 1806 may be utilized to align the prong 1806 with the target area accurately. In an example of use, a distance from an orifice 1870 of a cavity to a target area 1865 may be measured to determine an appropriate depth marking 1810 (e.g., 1810a and 1810b in the illustrated example) that will align the aperture 1805 of the prong 1806 with the target area 1865.

In an example embodiment, a proximal stopper/flange 1815 may be positioned at the depth marking 1810b to prevent the prong 1806 from being inserted into the cavity orifice 1870 past the depth marking 1810b. In some embodiments, the proximal stopper 1815 may be used with or without depth markings 1810 to stabilize the prong 1806 as it is inserted into the subject.

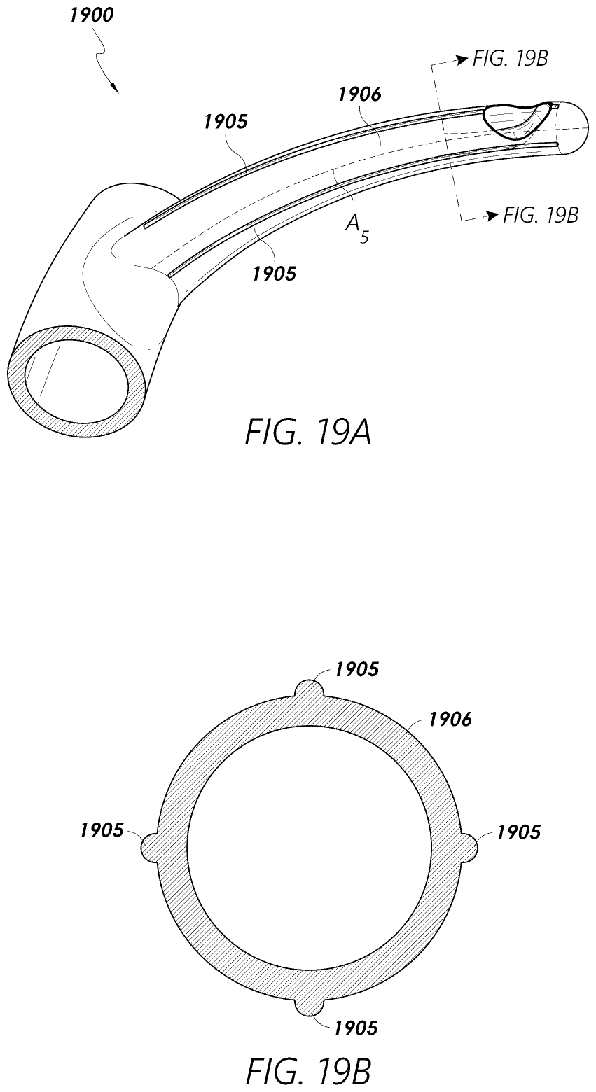

FIG. 19A is an illustration of an embodiment of a cannula 1900 comprising one or more prongs 1906 having one or more lengthwise protrusions 1905 projecting from an outer surface/diameter of the shaft of the prong 1906 along a length of the prong 1906 (i.e., in parallel with the longitudinal axis A₅ of the prong 1906). The one or more protrusions 1905, which may have the form of rib(s), curb(s), or similar structures, can serve a spacing/offsetting function such as to prevent the tubular wall of the prong 1906 from directly contacting anatomical walls or other features when temperature-controlled fluid is being passed through the prong 1906.

FIG. 19B shows a cross-sectional view of the prong 1906, as shown in FIG. 19A. FIG. 19B shows the protrusions 1905 as ribbed features that run in the axial dimension with respect to the longitudinal axis A₅ of the prong 1906. The one or more protrusions 1905 may allow the prong 1906 to be more easily inserted and retracted from the cavity by decreasing the frictional contact surface area of the prong 1906. The illustrated example includes four circumferentially-distributed protrusions 1905. Alternative embodiments may comprise more or fewer ribbed portions. For example, the prong 1906 may comprise two, six, eight, ten, or other number of ribs/projections, which may be integrally formed with the prong shaft or may be attached in some manner.

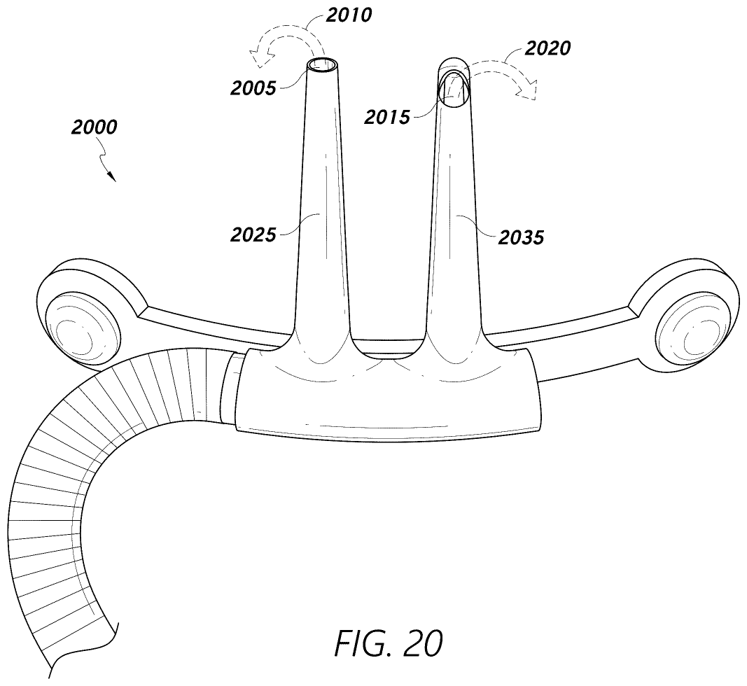

FIG. 20 shows an embodiment of the nasal cannula assembly 2000 that includes a first elongated prong 2025 with an aperture 2005 on the distal end of the first elongated prong 2025 and a second elongated prong 2035 with an aperture 2015 on the side of the second elongated prong 2035. Elongated prongs of various sizes and shapes may be exchanged on the nasal cannula assembly 2000 based on needs of the subject. In various embodiments, it may be advantageous to utilize a nasal cannula assembly 2000 with elongated prongs of different shapes, lengths, aperture sizes, or aperture locations. As shown in FIG. 20, the nasal cannula assembly 2000 includes two elongated prongs that are configured to direct a fluid in multiple directions within the cavity of the subject.

For example, it may be beneficial or advantageous to simultaneously direct fluid to two separate areas within a cavity. For instance, fluid may be directed toward a wall within a cavity while simultaneously being directed deeper into the cavity. In an example embodiment, fluid may be directed deeper into a cavity by the first elongated prong 2025 as denoted by the arrow 2010 while at the same time, fluid may be directed toward a side of the cavity by the second elongated prong 2035 as denoted by the arrow 2020. In an example of use, one elongated prong may be switched off and fluid directed through the other elongated prong. A direction of fluid through elongated prongs may be quickly alternated to modify physical characteristics of a target location for the fluid.

In an example of use, a temperature of a target location in a cavity a may be controlled by alternating a direction of fluid through one or more elongated prongs. For instance, a first elongated prong may be configured to direct fluid directly onto a target location while a second elongated prong may be configured to direct fluid further away from the target location. A the direction of temperature-controlled fluid through the elongated prongs may alternate to adjust the temperature at the target location without modifying the temperature of the fluid.

Provided below is a list of examples, each of which may include aspects of any of the other examples disclosed herein. Furthermore, aspects of any example described above may be implemented in any of the numbered examples provided below.

Example 1: A nasal cannula system, comprising a nasal cannula comprising a first elongated prong configured to be inserted into a cavity of a subject, the first elongated prong having a first axial fluid channel and a first aperture on a side wall of the first elongated prong, the first aperture providing access to the first axial fluid channel. The nasal cannual further comprises a temperature controller configured to regulate a temperature of a fluid prior to the fluid being directed through the first axial fluid channel and a fluid supply configured to direct the fluid through the first axial fluid channel of the first elongated prong and out of the first aperture to a target area.

Example 2: The nasal cannula system of any example herein, in particular example 1, wherein a distal end of the first elongated prong is sealed.

Example 3: The nasal cannula system of any example herein, in particular example 2, wherein the first aperture has a diameter that is larger than a diameter of the first elongated prong.

Example 4: The nasal cannula system of any example herein, in particular example 3, wherein the first elongated prong has a length configured to extend from nares of a subject to within about 2 to 3 cm of the target area, the target area being one of an internal carotid artery, a circle of willis, or a basal cistern of the subject.

Example 5: The nasal cannula system of any example herein, in particular example 4, wherein the length of the first elongated prong is five times larger than a distance between the first elongated prong and a second elongated prong.

Example 6: The nasal cannula system of any example herein, in particular example 1, wherein the nasal cannula further comprises a second elongated prong with a second aperture being located at a terminal end of the second elongated prong.

Example 7: The nasal cannula system of any example herein, in particular example 6, wherein the second elongated prong has a second aperture on a side wall of the second elongated prong.

Example 8: The nasal cannula system of any example herein, in particular example 1, further comprising a second elongated prong that comprises a temperature sensor electrically coupled to the temperature controller.

Example 9: The nasal cannula system of any example herein, in particular example 1, wherein the first elongated prong includes one or more depth markings on a proximal portion of the first elongated prong.

Example 10: The nasal cannula system of any example herein, in particular example 1, wherein the temperature controller comprises a thermoelectric device configured to cool fluid passed over a surface thereof.

Example 11: A nasal cannula comprising a fluid supply connector hub configured to connect to a fluid supply line, a first elongated prong comprising an axial fluid channel that is fluidly coupled to a lumen of the fluid supply connector hub, the first elongated prong projecting from the fluid supply connector hub and having a curved shape, and a fluid egress aperture positioned on a side of the first elongated prong opposite a curve of the first elongated prong.

Example 12: The nasal cannula of any example herein, in particular example 11, further comprising a second elongated prong that projects from the fluid supply connector hub and runs parallel with the first elongated prong.

Example 13: The nasal cannula of any example herein, in particular example 12, wherein a length of the first elongated prong is more than twice a distance between an axis of the first elongated prong and an axis of the second elongated prong.

Example 14: The nasal cannula of any example herein, in particular example 13, wherein the first elongated prong has a length of the first elongated prong is five times larger than a distance between the first elongated prong and the second elongated prong.

Example 15: The nasal cannula of any example herein, in particular example 11, wherein a proximal portion of the first elongated prong includes a plurality of depth markings.

Example 16: The nasal cannula of any example herein, in particular example 15, further comprising a proximal flange that is configured to axially slide along at least a portion of the proximal portion of the first elongated prong to set a proximal stop position of the nasal cannula.

Example 17: The nasal cannula of any example herein, in particular example 11, wherein a line normal to the fluid egress aperture at a center of the fluid egress aperture is angled relative to an angle of an axis of the first elongated prong at a proximal base of the first elongated prong by an angle of less than 90°.

Example 18: The nasal cannula of any example herein, in particular example 11, wherein the fluid egress aperture has a triangular, oval, circular, elliptical, square, rectangular, diamond or star shape.

Example 19: The nasal cannula of any example herein, in particular example 11, wherein the first elongated prong is detachable from the fluid supply connector hub.

Example 20: The nasal cannula of any example herein, in particular example 11, further comprising a plurality of rib projections that run along a length of the first elongated prong to create a gap and reduce friction between tissue and the nasal cannula.

Example 21: A temperature control system, comprising a nasal cannula comprising a first elongated prong having a first axial fluid channel, an aperture on a side wall of the first elongated prong, the first aperture providing access to the first axial fluid channel, and a second elongated prong having a temperature sensor associated with a distal portion thereof. The temperature control system further comprises a fluid supply configured to direct fluid through the first axial fluid channel of the first elongated prong and out of the first aperture to a target area, and control circuitry configured to receive one or more signals from the temperature sensor and modify one or more properties of the fluid in response to the one or more signals from the temperature sensor.

Example 22: The temperature control system of any example herein, in particular example 21, wherein the one or more properties of the fluid modified by the control circuitry comprise at least one of fluid temperature or fluid flow rate.

Example 23: The temperature control system of any example herein, in particular example 21, wherein a distal end of the first elongated prong is sealed.

Example 24: The temperature control system of any example herein, in particular example 21, wherein said modifying the one or more properties of the fluid involves modifying a temperature of the fluid.

Example 25: The temperature control system of any example herein, in particular example 21, further comprising an electrical conductor electrically connected between the temperature sensor and the control circuitry, the electrical conductor running a length of the second elongated prong.

Example 26: The temperature control system of any example herein, in particular example 21, further comprising a blood oxygen sensor associated with the second elongated prong.

Example 27: The temperature control system of any example herein, in particular example 21, wherein said modifying the one or more properties of the fluid involves modifying a flow rate of the fluid.

Example 28: The temperature control system of any example herein, in particular example 21, wherein said modifying the one or more properties of the fluid involves modifying a composition of the fluid.

Example 29: The temperature control system of any example herein, in particular example 21, wherein the control circuitry is configured to control a thermoelectric device configured to cool fluid passed over a surface thereof.

Example 30: The temperature control system of any example herein, in particular example 21, wherein the first elongated prong has a length of between about 2.0 inches and about 2.6 inches along a longitudinal axis of the first elongated prong.

Example 31: The temperature control system of any example herein, in particular example 21, wherein both the first elongated prong and the second elongated prong have a length that is at least twice a lateral distance between axes of the first elongated prong and the second elongated prong.

Example 32: A nasal cannula comprising a fluid supply connector hub configured to connect to a fluid supply line, a first elongated prong comprising an axial fluid channel that is fluidly coupled to a lumen of the fluid supply connector hub, the first elongated prong projecting from the fluid supply connector hub and having a curved shape, a fluid egress aperture positioned on a side wall of the first elongated prong opposite a curve of the first elongated prong, and a second elongated prong projecting from the fluid supply connector hub and comprising a sensor.

Example 33: The nasal cannula of any example herein, in particular example 32, wherein the sensor is coupled to a distal portion of the second elongated prong and electrical wiring runs along a length of the second elongated prong and connects to the sensor.

Example 34: The nasal cannula of any example herein, in particular example 32, wherein a length of the first elongated prong is more than twice a distance between an axis of the first elongated prong and an axis of the second elongated prong.

Example 35: The nasal cannula of any example herein, in particular example 34, wherein the first elongated prong and the second elongated prong have a common length.

Example 36: The nasal cannula of any example herein, in particular example 34, wherein the second elongated prong is shorter than the first elongated prong.

Example 37: The nasal cannula of any example herein, in particular example 34, wherein the length of the first elongated prong is at least five times greater than the distance between the axis of the first elongated prong and the axis of the second elongated prong.

Example 38: The nasal cannula of any example herein, in particular example 32, wherein a proximal portion of the first elongated prong includes a plurality of depth markings.

Example 39: The nasal cannula of any example herein, in particular example 38, further comprising a proximal flange that is configured to axially slide along at least a portion of the proximal portion of the first elongated prong to set a proximal stop position of the nasal cannula.

Example 40: The nasal cannula of any example herein, in particular example 32, wherein the sensor comprises a temperature sensor.

Example 41: A method for controlling tissue temperature, the method comprising providing a nasal cannula comprising a first elongated prong having an axial fluid channel and a downwardly-curved shape and a first aperture on a side wall of a distal portion of the first elongated prong, the first aperture providing access to the axial fluid channel. The method further comprises inserting the first elongated prong into a nasal cavity of a subject to face the first aperture at a pharyngeal wall of the subject, and modifying a temperature of the pharyngeal wall by delivering a first fluid portion through the axial fluid channel and out of the first aperture to the pharyngeal wall.

Example 42: The method of any example herein, in particular example 41, wherein a distal end of the first elongated prong is sealed.

Example 43: The method of any example herein, in particular example 41, further comprising delivering a second fluid portion through the axial fluid channel and out of a distal axial aperture of the first elongated prong into a larynx of the subject.

Example 44: The method of any example herein, in particular example 41, wherein the first elongated prong has a length configured to extend from nares of the subject to within 2 cm of the pharyngeal wall.

Example 45: The method of any example herein, in particular example 1, wherein the nasal cannula further comprises a second elongated prong.

Example 46: The method of any example herein, in particular example 45, wherein the second elongated prong has a second aperture on a side wall of the second elongated prong.

Example 47: The method of any example herein, in particular example 45, wherein the second elongated prong has a temperature sensor associated with a distal portion thereof.

Example 48: The method of any example herein, in particular example 47, further comprising modifying a temperature of the first fluid portion based on a measurement from the temperature sensor.

Example 49: The method of any example herein, in particular example 47, further comprising modifying a flow rate of the first fluid portion based on a measurement from the temperature sensor.

Example 50: The method of any example herein, in particular example 45, wherein a length of the first elongated prong is at least twice a distance between the first elongated prong and the second elongated prong.

Example 51: A method for controlling a temperature of tissue, the method comprising providing a nasal cannula system comprising a first elongated prong having an axial fluid channel, an aperture on a side wall of the first elongated prong, the first aperture providing access to the axial fluid channel, a second elongated prong having a temperature sensor associated with a distal portion thereof, and a fluid supply configured to direct fluid through the axial fluid channel of the first elongated prong and out of the first aperture to a target area. The method further comprises receiving, by a control circuitry, one or more signals from the temperature sensor and modifying, by the control circuitry one or more properties of the fluid in response to the one or more signals from the temperature sensor.

Example 52: The method of any example herein, in particular example 51, wherein modifying the one or more properties of the fluid comprises modifying least one of fluid temperature or fluid flow rate.

Example 53: The method of any example herein, in particular example 51, wherein modifying the one or more properties of the fluid involves modifying a temperature of the fluid.

Example 54: The method of any example herein, in particular example 51, further comprising transmitting an electrical signal between the temperature sensor and the control circuitry.

Example 55: The method of any example herein, in particular example 51, wherein the measurement is selected from at least one of the group consisting of a temperature, a blood-oxygen level, a respiration rate, a blood pressure, and a heart rate.

Example 56: The method of any example herein, in particular example 51, wherein delivering the fluid comprises directing a surface normal of an outside area of the aperture toward a target area.

Example 57: The method of any example herein, in particular example 51, wherein a length of the first elongated prong is about five times larger than a distance between the first elongated prong and the second elongated prong.

Example 58: The method of any example herein, in particular example 57, wherein the first elongated prong has a length of between about 2.0 inches and about 2.6 inches along a longitudinal axis of the first elongated prong.

Example 59: The method of any example herein, in particular example 58, wherein both the first elongated prong and the second elongated prong have a length that is at least twice a lateral distance between axes of the first elongated prong and the second elongated prong.

Example 60: The method of any example herein, in particular example 61, wherein the second elongated prong is shorter than the first elongated prong.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or blocks. Thus, such conditional language is not generally intended to imply that features, elements and/or blocks are in any way required for one or more embodiments or that one or more embodiments necessarily include logic for deciding, with or without author input or prompting, whether these features, elements and/or blocks are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for the use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It should be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method for controlling tissue temperature, the method comprising:
providing a nasal cannula comprising:
a first elongated prong having an axial fluid channel and a downwardly-curved shape; and
a first aperture on a side wall of a distal portion of the first elongated prong, the first aperture providing access to the axial fluid channel;
inserting the first elongated prong into a nasal cavity of a subject to face the first aperture at a pharyngeal wall of the subject; and
modifying a temperature of the pharyngeal wall by delivering a first fluid portion through the axial fluid channel and out of the first aperture to the pharyngeal wall.

2. The method of claim 1, wherein a distal end of the first elongated prong is sealed.

3. The method of claim 1, further comprising delivering a second fluid portion through the axial fluid channel and out of a distal axial aperture of the first elongated prong into a larynx of the subject.

4. The method of claim 1, wherein the first elongated prong has a length configured to extend from nares of the subject to within 2 cm of the pharyngeal wall.

5. The method of claim 1, wherein the nasal cannula further comprises a second elongated prong.

6. The method of claim 5, wherein the second elongated prong has a second aperture on a side wall of the second elongated prong.

7. The method of claim 5, wherein the second elongated prong has a temperature sensor associated with a distal portion thereof.

8. The method of claim 7, further comprising modifying a temperature of the first fluid portion based on a measurement from the temperature sensor.

9. The method of claim 7, further comprising modifying a flow rate of the first fluid portion based on a measurement from the temperature sensor.

10. The method of claim 5, wherein a length of the first elongated prong is at least twice a distance between the first elongated prong and the second elongated prong.

11. A method for controlling a temperature of tissue, the method comprising:
providing a nasal cannula system comprising:
a first elongated prong having an axial fluid channel;
an aperture on a side wall of the first elongated prong, the first aperture providing access to the axial fluid channel;
a second elongated prong having a temperature sensor associated with a distal portion thereof; and
a fluid supply configured to direct fluid through the axial fluid channel of the first elongated prong and out of the first aperture to a target area;
receiving, by a control circuitry, one or more signals from the temperature sensor; and
modifying, by the control circuitry one or more properties of the fluid in response to the one or more signals from the temperature sensor.

12. The method of claim 11, wherein modifying the one or more properties of the fluid comprises modifying least one of fluid temperature or fluid flow rate.

13. The method of claim 11, wherein modifying the one or more properties of the fluid involves modifying a temperature of the fluid.

14. The method of claim 11, further comprising transmitting an electrical signal between the temperature sensor and the control circuitry.

15. The method of claim 11, wherein the measurement is selected from at least one of the group consisting of a temperature, a blood-oxygen level, a respiration rate, a blood pressure, and a heart rate.

16. The method of claim 11, wherein delivering the fluid comprises directing a surface normal of an outside area of the aperture toward a target area.

17. The method of claim 11, wherein a length of the first elongated prong is about five times larger than a distance between the first elongated prong and the second elongated prong.

18. The method of claim 17, wherein the first elongated prong has a length of between about 2.0 inches and about 2.6 inches along a longitudinal axis of the first elongated prong.

19. The method of claim 18, wherein both the first elongated prong and the second elongated prong have a length that is at least twice a lateral distance between axes of the first elongated prong and the second elongated prong.

20. The method of claim 11, wherein the second elongated prong is shorter than the first elongated prong.
